(19) United States
(12) Patent Application Publication
(10) Pub. No.: US 2023/0190653 A1
(43) Pub. Date: Jun. 22, 2023

(54) METHOD FOR REDUCING OFF-TARGET UPTAKE OR ACCUMULATION OF AGENTS

(71) Applicant: The Regents of the University of Colorado, A Body Corporate, Aurora, CO (US)

(72) Inventors: Thomas Anchordoquy, Lakewood, CO (US); Jamie Betker, Denver, CO (US); Scott Tilden, Thornton, CO (US)

(21) Appl. No.: 18/084,105

(22) Filed: Dec. 19, 2022

Related U.S. Application Data
(60) Provisional application No. 63/416,706, filed on Oct. 17, 2022.
(60) Provisional application No. 63/291,142, filed on Dec. 17, 2021.

(51) Int. Cl. A61K 9/127; A61K 45/06; A61P 35/00

(52) U.S. Cl. CPC A61K 9/1272; A61K 45/06; A61P 35/00

(57) ABSTRACT

Described herein are compositions and methods for reducing off-target deposition of therapeutic and/or imaging agents in a subject with cancer comprising administering to the subject a predose of an interferon composition and/or a nucleic acid and a nanoparticle mimic, e.g., a lipoplex composition or a virus-like particle, such that epithelial tightening occurs in nontumor tissue of the subject thereby reducing uptake or accumulation of the therapeutic and/or imaging agent in off-target, nontumor tissue compared to tumor tissue.

### CROSS-REFERENCE TO RELATED APPLICATIONS

This application claims priority to U.S. Provisional Applications 63/291,142 filed on Dec. 17, 2021 and 63/416,706, filed on Oct. 17, 2002, which are incorporated herein by reference in their entirety.

### BACKGROUND

Almost one million patients receive chemotherapy in the United States each year; approximately 10% of the worldwide demand for chemotherapy. Furthermore, the demand for chemotherapy is expected to increase by over 50% by 2040. It is well established that the significant adverse effects associated with this treatment are due to drug accumulation in off-target sites, and that repeat administration is required to achieve therapeutic levels in the tumor. A typical course of chemotherapy involves daily infusion (3-5 times/week) for six weeks, and this course of therapy is often repeated multiple times. This prolonged treatment is necessary because the toxicity of the chemotherapeutic drug limits dosing to what can be tolerated by the patient. Even under these conditions, the amount of drug a patient can receive over a lifetime is limited due to concerns about organ toxicity. It follows that reducing accumulation in nontumor tissues would permit more aggressive dosing that could improve patient outcomes. Considering the extent to which patients are subjected to multiple rounds of chronic administration, even a modest reduction in off-target accumulation from each dose would have significant benefits to the millions of patients undergoing chemotherapy each year.

Therefore, there is a need for a treatment method that reduces off-target accumulation of chemotherapeutic drugs in order to reduce toxicity to the patient.

### BRIEF SUMMARY

In an aspect, a method for treating cancer in a subject comprises predosing/pretreating the subject to induce tightening of epithelial junctions in nontumor tissue, followed by administering a dose of an anti-cancer agent, wherein the predosing/pretreating increases uptake or accumulation of the anti-cancer agent in tumor tissue compared to uptake or accumulation of the anti-cancer agent in nontumor tissue.

In another aspect, a method for treating cancer in a subject comprises predosing the subject to induce tightening of epithelial junctions in nontumor tissue prior to administering a dose of drug or imaging agent such that the drug or imaging agent administered following the predosing accumulates or is taken up in tumor tissue compared to nontumor tissue, wherein predosing is with an effective amount of a nucleic acid and a nanoparticle mimic. The nucleic acid can be encapsulated within or absorbed to a nanoparticle mimic. A nanoparticle mimic provides the delivery function of organisms or particles such as, but not limited to, pathogens, viruses, bacteria, fungus, parasites, prions, and cells. The nanoparticle mimic can comprise polymers, proteins, carbohydrates, lipids, viruses, and the like. The nanoparticle can be a virus-like particle or a lipid nanoparticle.

In another aspect, a method of treating cancer in a subject comprises predosing the subject with an effective amount of a lipoplex composition comprising a lipid nanoparticle (NP) and a nucleic acid, wherein the predosing is effective to induce tightening of epithelial junctions in nontumor tissue, and administering a therapeutically effective amount of an anti-cancer agent, wherein predosing provides uptake or accumulation of the anti-cancer agent in tumor tissue compared to uptake or accumulation of the anti-cancer agent in nontumor tissue.

In another aspect, a method of treating cancer in a subject comprises predosing the subject with an effective amount of interferon lambda (IFN-λ), wherein the predosing is effective to induce tightening of epithelial junctions in nontumor tissue and administering a therapeutically effective amount of an anti-cancer agent, wherein predosing provides uptake or accumulation of the anti-cancer agent in tumor tissue compared to uptake or accumulation of the anti-cancer agent in nontumor tissue.

In another aspect, a method for selectively reducing uptake or accumulation in nontumor tissue of a patient while enhancing deposition or delivery of an agent in a tumor comprises administering to said patient an effective amount of a lipoplex composition comprising a lipid NP or a virus-like particle (VLP) and a nucleic acid, wherein the effective amount is effective for inducing tightening of epithelial junctions in nontumor tissue, and reducing accumulation or uptake in nontumor tissue while enhancing deposition or delivery of the agent in a tumor.

In another aspect, a method for selectively reducing accumulation or uptake in nontumor tissue of a patient while enhancing deposition or delivery of an agent in a tumor comprises administering to said patient an effective amount of IFN-λ, wherein the effective amount is effective for inducing tightening of epithelial junctions in nontumor tissue and reducing accumulation or uptake in nontumor tissue while enhancing deposition or delivery of the agent in a tumor.

In another aspect, a method for improving imaging of tumors in a subject, comprises predosing the subject with an effective amount of a lipoplex composition comprising a lipid nanoparticle and a nucleic acid, wherein the predosing is effective to induce tightening of epithelial junctions in nontumor tissue, and administering an imaging agent, wherein the predosing increases uptake or accumulation of the imaging agent in tumor tissue compared to uptake or accumulation of the imaging agent in nontumor tissue.

In another aspect, a method for improving imaging of tumors in a subject, comprises predosing the subject with an effective amount of IFN-λ, wherein the predosing is effective to induce tightening of epithelial junctions in nontumor tissue, and administering an imaging agent, wherein the predosing increases accumulation, uptake or deposition of the imaging agent in tumor tissue compared to accumulation, uptake or deposition of the imaging agent in nontumor tissue.

### BRIEF DESCRIPTION OF THE DRAWINGS

FIGS. 1A-B show lipoplex levels in blood 1 h after repeated intravenous injections. Plasmid levels were quantified in the plasma (black bars) and cell fraction (shaded bars) after repeat administration of lipoplexes coated with lactose (1A) or PEG (1B). Each bar represents the mean±one standard error of measurements on triplicate mice.

FIGS. 2A-D show total plasmid accumulation in tissues. Quantitative PCR was used to monitor the accumulation of plasmid per gram tissue in the liver (2A), spleen (2B), lung (2C), and tumor (2D) 24 and 72 h after each intravenous injection of lactosylated (black bars) or PEGylated (shaded bars) lipoplexes. Note the different scales used in the y-axes. Each bar represents the mean±one standard error of measurements on organs from triplicate mice.

FIGS. 3A-D show reporter gene expression. Luciferase expression per gram tissue is depicted for the liver (3A), spleen (3B), lung (3C), and tumor (3D) 24 and 72 h after each intravenous injection of lactosylated (black bars) or PEGylated (shaded bars) lipoplexes. Note the different scales used in the y-axes. Each bar represents the mean±one standard error of measurements on organs from triplicate mice.

FIGS. 4A-C show cytokine levels in the blood. IFNγ (4A), IL-6 (4B), and TNFα (4C) were quantified in plasma 24 and 72 h after each intravenous injection. Each bar represents the mean±one standard error of measurements (per mL plasma) on triplicate mice injected with saline (PBS; black bars) or lipoplexes coated with either lactose (light shading) or PEG (dark shading).

FIGS. 5A-D show IFN γ levels in the liver (5A), spleen (5B), lung (5C) and tumor (5D) were quantified in tissues extracted 24 and 72 h after each intravenous injection. Each bar represents the mean±one standard error of measurements (per gram tissue) on triplicate mice injected with saline (PBS; black bars) or lipoplexes coated with either lactose (light shading) or PEG (dark shading).

FIGS. 6A-D show IL-6 levels in the liver (6A), spleen (6B), lung (6C) and tumor (6D) were quantified in tissues extracted 24 and 72 h after each intravenous injection. Each bar represents the mean±one standard error of measurements (per gram tissue) on triplicate mice injected with saline (PBS; black bars) or lipoplexes coated with either lactose (light shading) or PEG (dark shading).

FIGS. 7A-D show TNFα levels in the liver (7A), spleen (7B), lung (77C) and tumor (D) were quantified in lungs extracted 24 and 72 h after each intravenous injection. Each bar represents the mean±one standard error of measurements (per gram tissue) on triplicate mice injected with saline (PBS; black bars) or lipoplexes coated with either lactose (light shading) or PEG (dark shading).

FIGS. 8A-D show expression efficiency in tissues. Luciferase expression in liver (8A), spleen (8B), lungs (8C) or tumor (8D) is standardized against the amount of plasmid to depict the efficiency with which plasmids are expressed 24 and 72 h after each intravenous injection. Each bar represents the mean±one standard error of measurements on triplicate mice injected with lipoplexes coated with either 5% lactose (black) or PEG (shaded).

FIG. 9 shows in vitro expression efficiency in CT26 cells. Luciferase expression in CT26 cells in culture was standardized against the amount of plasmid (assessed by qPCR by using 3 technical replicates per each of the three triplicate samples as is recommended by the manufacturer) to depict the efficiency after a single lipoplex treatment. Error was estimated by using the Taylor series expansion on both the qPCR and luminescence measurements. Each bar represents the mean±one error of measurements on triplicate wells of cells incubated with uncoated lipoplexes (black bars) or lipoplexes coated with either 5% lactose (light shading) or PEG (dark shading).

FIG. 10 shows FITC-dextran fluorescence (Excitation: 490 nm, Emission: 520 nm) in 1×PBS (pH 8.25) standard curve. Each point represents the mean value of 6 measured samples. Error bars represent standard deviation.

FIG. 11 shows liver, spleen, lung, brain, heart, kidney and tumor dextran extraction efficiency standard curves. Each point represents the average value of duplicate samples.

FIGS. 12A-G show total dextran accumulation in major organ and tumor tissues 24 hours after PBS injection (control) or Lipoplex injection (tightened) in Balb/c female mice. (Statistical significance=p<0.05, ns=nonsignificant). (12A) Liver dextran accumulation. (12B) Spleen dextran accumulation. (12C) Tumors dextran accumulation. (12D) Lung dextran accumulation. (12E) Brain dextran accumulation. (12F) Heart dextran accumulation. (12G) Kidney dextran accumulation.

FIGS. 13A-F show ratio of tumor dextran accumulation to organ dextran accumulation in PBS (control) or Lipoplex (tightened) treated female Balb/c mice. (Statistical significance=p<0.05, ns=nonsignificant). (13A) Tumor:Liver accumulation ratio. (13B) Tumor:Spleen accumulation ratio. (13C) Tumor:Lung accumulation ratio. (13D) Tumor:Kidney accumulation ratio. (13E) Tumor:Heart accumulation ratio. (13F) Tumor:Brain accumulation ratio.

FIGS. 14A-G show total dextran accumulation in major organ and tumor tissues 24 hours after PBS (control) injection or Liposome injection in Balb/c female mice. (Statistical significance=p<0.05, ns=nonsignificant). (14A) Liver dextran accumulation. (14B) Spleen dextran accumulation. (14C) Tumor dextran accumulation. (14D) Kidney dextran accumulation. (14E) Brain dextran accumulation. (14F) Heart dextran accumulation. (14G) Lung dextran accumulation.

FIGS. 15A-G show total dextran accumulation in major organ and tumor tissues 24 hours after Liposome (−DNA) injection or Lipoplex (+DNA) injection in Balb/c female mice. (Statistical significance=p<0.05, ns=nonsignificant). (15A) Liver dextran accumulation. (15B) Spleen dextran accumulation. (15C) Tumor dextran accumulation. (15D) Lung dextran accumulation. (15E) Heart dextran accumulation. (15F) Brain dextran accumulation. (15G) Kidney dextran accumulation.

FIG. 16 shows total dextran accumulation in tumor tissues after PBS (Cntrl) or Lipoplex pre-treatment plus 1, 2, and 3 injections of dextran in female Balb/c mice (Statistical significance=p<0.05).

FIGS. 17A-D shows results of repeat injections of PEG-coated lipoplexes to mice bearing CT26 tumors. Plasmid levels in the major organs are not increased by repeat intravenous injection. However, plasmid levels in the tumor are enhanced by greater than two-fold after a second injection. Each bar represents the mean and standard error of tissues from 3 mice.

FIGS. 18A-D show tissue cytokine levels 24 h after the first intravenous injection of lipoplexes (as compared to saline) in Balb/c mice bearing CT26 tumors. Each bar represents the mean and standard error of tissues from 3 mice. Note the log scale on the y-axis.

FIGS. 19A-D show tissue cytokine levels 24 h after the second intravenous injection of lipoplexes (as compared to saline) in Balb/c mice bearing CT26 tumors. Each bar represents the mean and standard error of tissues from 3 mice. Note the log scale on the y-axis.

FIG. 20 is an MRI of tightening. Tumor-bearing mice were injected with PBS or lipoplex prior to treatment with ferumoxytol. ΔT2 values were mapped for tumor, spleen, and kidney, and demonstrate significant reductions in spleen and kidney accumulation. In contrast, tumor accumulation of ferumoxytol was unaffected. n=4.

FIGS. 21A-D show IFN-λ response. (21A) Quantification of IFN-λ in serum of Balb/c female mice after lipoplex, liposome, and 5% dextrose treatment using ELISA. Lipoplex treatment n−3-4. Liposome treatment n=4. Control treatment n=2. (21B) Quantification of IFN-λ in serum of Balb/c female mice after lipoplex treatment. (21C) Quantification of IFN-λ in serum of Balb/c female mice after liposome treatment. (21D) Quantification of IFN-λ in serum of Balb/c female mice after 5% dextrose (control) treatment. Error bars represent the standard error of the mean. Lower limit of quantification (LLoQ) of ELISA: 31.3 pg/mL. Lower limit of detection (LLoD) of ELISA: 3.9 pg/mL.

FIG. 22 shows a doxorubicin fluorescence (Excitation 470 nm, Emission: 595 nm) in 90% Isopropyl Alcohol/0.075 M HCl standard curve.

FIG. 23 shows doxorubicin fluorescence in supernatants of organs homogenized in 90% Isopropyl Alcohol/0.075 M HCl (Excitation: 470 nm, Emission: 595 nm). All y-axis represent fluorescence. Each point represents the average measured fluorescence of duplicated samples.

FIG. 24 shows a comparison of Doxil® accumulation in major organs and tissues of tumor bearing Balb/c female mice 24 hours after administration. Each x-axis represents the pre-treatment (tail-vein administration of 100 μL 1×PBS or 1 μg of IFN-λ in 100 μL 1×PBS) that the mice received 24 hours before Doxil® treatment. (*=p<0.05, **=p<0.005, ns=nonsignificant).

The above-described and other features will be appreciated and understood by those skilled in the art from the following detailed description, drawings, and appended claims.

### DETAILED DESCRIPTION

Described herein are methods of reducing uptake/accumulation of drugs or agents in off-target tissues compared to target tissues. Instead of engineering a delivery system that exhibits greater accumulation in the tumor, the methods of the invention are based on the novel and counterintuitive approach focusing on restricting uptake by nontumor tissues compared to tumor tissue. This basic approach has not been previously investigated due to the inability to preferentially reduce accumulation in only nontumor tissues. As shown in the Examples below, the data illustrate that epithelial tightening can be elicited in response to predosing by administration of a composition comprising nanoparticle mimics such as lipoplexes comprising lipid nanoparticles (NP) and nucleic acids, or by administration of IFN-λ. The epithelial tightening observed significantly reduces uptake in the major nontumor organs after lipoplex or IFN-λ pre-treatment. Curiously, this phenomenon is not observed when liposomes or other therapeutics are dosed in a similar manner. This mechanism of enhancing barrier integrity in nontumor tissue has the potential to significantly reduce off-target distribution/uptake or accumulation of administered drugs or imaging agents, allowing more aggressive dosing and markedly improved tumor treatment and/or imaging.

In an aspect, a method for treating cancer in a subject comprises predosing the subject to induce tightening of epithelial junctions in nontumor tissue prior to administering a dose of drug or imaging agent such that the drug or imaging agent administered following the predosing accumulates or is otherwise taken up in tumor tissue but not in nontumor tissue, wherein predosing comprises administering an effective amount of a nucleic acid and a nanoparticle mimic. The nucleic acid can be encapsulated within or absorbed to the nanoparticle mimic. A nanoparticle mimic can mimic the delivery function of organisms or particles such as, but not limited to, pathogens, viruses, bacteria, fungus, parasites, prions, and cells. The nanoparticle mimic can comprise polymers, proteins, carbohydrates, lipids, or viruses. The nanoparticle can be a virus-like particle or a lipid nanoparticle.

In another aspect, a method for treating cancer in a subject comprises predosing a subject with an IFN-λ composition to induce tightening of epithelial junctions in nontumor tissue prior to administering a dose of a drug or imaging agent such that the drug or imaging agent administered following the predosing accumulates or is otherwise taken up in tumor tissue compared to nontumor tissue.

IFN-λs, designated as type III interferons, are a newly described group of cytokines that consist of IFN-2d, 2, 3 (also referred to as interleukin-29, 28A, and 28B, respectively), that are genetically encoded by three different genes located on chromosome 19. At the protein level, IFN-λ2 and -λ3 are highly homologous, with 96% amino acid identity, while IFN-λ1 shares approximately 81% homology with IFN-λ2 and -λ3. IFN-λs activate signal transduction via the JAK/STAT pathway similar to that induced by type I IFN, including the activation of JAK1 and TYK2 kinases, the phosphorylation of STAT proteins, and the activation of the transcription complex of IFN-stimulated gene factor 3 (ISGF3).

IFN-λs signal through a heterodimeric receptor complex consisting of unique IFN-λ receptor 1 (IFN-λR1) and IL-10 receptor 2 (IL-10R2). As previously reported, IFN-λR1 has a very restricted expression pattern with the highest levels in epithelial cells, melanocytes, and hepatocytes, and the lowest level in primary central nervous system (CNS) cells. Blood immune system cells express high levels of a short IFN-λ receptor splice variant (sIFN-λR1) that inhibits IFN-λ action.

IFN-λs display structural features similar to IL-10-related cytokines, but functionally possess type I IFN-like anti-viral and anti-proliferative activity. IFN-λ1 and -λ2 have been demonstrated to reduce viral replication or the cytopathic effect of various viruses, including DNA viruses (hepatitis B virus and herpes simplex virus 2), ss (+) RNA viruses (EMCV) and hepatitis C virus, ss (—) RNA viruses (vesicular stomatitis virus) and influenza-A virus and double-stranded RNA viruses, such as rotavirus. IFN-λ3 has been identified from genetic studies as a key cytokine in HCV infection, and has also shown potent activity against EMCV. A deficiency of rhinovirus-induced IFN-λ production was reported to be highly correlated with the severity of rhinovirus-induced asthma exacerbation and IFN-λ therapy has been suggested as a new approach for treatment of allergic asthma.

In an aspect, the IFN-λ is IFN-λ1, IFN-λ2, or IFN-λ3, or a combination thereof. The IFN-λ can be a recombinant form of IFN-λ, can be PEGylated, or conjugated or complexed with a targeting agent or moiety that specifically binds to the target tissue or cancer cell or a tumor antigen on a cancer cell, or a cell surface receptor that is a growth factor, a hormone, an extracellular matrix molecule such as transforming growth factor (TGF), epidermal growth factor (EGF), insulin-like growth factor (IGF), heregulin, platelet-derived growth factor (PDGF), vascular endothelial growth factor (VEGF), hypoxia inducible factor (HIT), c-Met, human chorionic gonadotropin, gonadotropin releasing hormone, androgen, estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, collagen, elastin, biglycan, decorin, lumican, versican, perlecan, C-reactive protein, ApoE, and laminins.

In some embodiments, the targeting moiety specifically conjugated to IFN-λ binds to a tumor antigen selected from the group consisting of EGFR mutant, HER2/neu, HER3, HER4, CD4, CD19, CD20, CD22, CD29b, CD30, CD33, CD37, CD38, CD52, CD70, CD79b, CD123, CD138, CD200, CD276, CXCR3, CXCR5, CCR3, CCR4, CCR9, CRTH2, PMCH, endoplasmin, CS1, CEA, mesothelin, G250, MUC1, MUC16, PSMA, ADAM17, EPCAM, EphA2, MCSP, GPA33, NAPi2b, STEAP1, CEACAM1, CEACAM5, GPNMB and TROP.

The targeting agent may be an antibody or portion of an antibody, e.g., a C-terminus, N-terminus, light chain, heavy chain, or Fab fragment of an antibody. The IFN-λ may be used with other agents, e.g., other interferons, or a nucleic acid/nanoparticle mimic, or a virus-like particle, or a lipoplex. When used with other agents, the IFN-λ may be administered prior to, concurrently with, or after the other agent. When administered concurrently, the IFN-λ may be either conjugated to or separate from the other agent.

Dosages of one or more IFN-λ or a pharmaceutical composition comprising the one or more IFN-λ, are typically in the microgram range, for example 180 μg s.c. once per week, or 100 to 189 μg, or 135 μg, or 90 μg, or 250 μg s.c. every other day, depending on the size, age, weight, and response of the subject to the interferon.

In an aspect, a method for treating cancer in a subject comprises predosing the subject with a lipoplex composition to induce tightening of epithelial junctions in nontumor tissue prior to administering a dose of a drug or an imaging agent such that the drug or imaging agent administered following the predosing accumulates or is otherwise taken up in tumor tissue compared to nontumor tissue.

In an aspect, lipoplex compositions described herein comprise a lipid nanoparticle (NP) and a nucleic acid. The lipid composition in a nanoparticle composition such as a lipid nanoparticles (LNPs), liposomes (e.g., lipid vesicles), micelles, dendrimers, lipidoids, virus-like particles, and lipoplexes can be influenced by the selection of the lipid component, the degree of lipid saturation, the nature of the PEGylation, ratio of all components and biophysical parameters such as size. In some aspects, a lipoplex composition comprises an asymmetric phospholipid, e.g., sphingosine, from about 20 to about 45%, from about 40% to about 50%, from about 50% to about 60% and/or from about 55% to about 65%.

In an aspect, the lipid NP comprises a symmetric phospholipid, e.g., DAPC, at a molar ratio of about 20-70%, about 10-60%, about 20-55%.

In another aspect, the lipid NP comprises a sterol, e.g., cholesterol, at a molar ratio of about and 0.5-40%, about 5-25%.

In other aspects, the nanoparticle comprises a polyethylene glycol (PEG), e.g., a PEG-modified lipid, at a molar ratio of about 1-10%, at about 0.5-15%, at about 2-8%.

In some aspects, the ratio of lipid to nucleic acid in the lipoplexes is from about 0.1-1 N/P ratio (ratio of positively-charged polymer amine (N=nitrogen) groups to negatively-charged nucleic acid phosphate (P)), from about 0.3-0.8, from about 0.4-0.7, or from about 0.5-0.6 N/P ratio.

### Lipid Nanoparticle Components

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

### A. Phospholipids

The lipid composition of the NP disclosed herein can comprise one or more phospholipids, for example, one or more saturated or (poly)unsaturated phospholipids or a combination thereof. In general, phospholipids comprise a phospholipid moiety and one or more fatty acid moieties. A phospholipid moiety may be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety may be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids may facilitate fusion to a membrane. For example, a cationic phospholipid may interact with one or more negatively charged phospholipids of a membrane (e.g., a cellular or intracellular membrane). In one aspect, the fusion of a phospholipid to a membrane may elicit tightening. In one aspect, fusion is to an immune cell responsible for the tightening effect, and one or more elements (e.g., a therapeutic agent, a tightening agent, an immune agent) is allowed to pass through the membrane permitting delivery of the one or more elements to an immune cell.

Non-natural phospholipid species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin. In some aspects, a nanoparticle disclosed herein can comprise more than one phospholipid. When more than one phospholipid is used, such phospholipids can belong to the same phospholipid class (e.g., MSPC and DSPC) or different classes (e.g., MSPC and MSPE).

Phospholipids may be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some aspects, the lipid composition of a nanoparticle disclosed herein comprises at least one asymmetric phospholipid. Asymmetric phospholipids may be selected from the non-limiting group consisting of 1-myristoyl-2-palmitoyl-sn-glycero-3-phosphocholine (14:0-16:0 PC, MPPC), 1-myristoyl-2-stearoyl-sn-glycero-3-phosphocholine (14:0-18:0 PC, MSPC), 1-palmitoyl-2-acetyl-sn-glycero-3-phosphocholine (16:0-02:0 PC), 1-palmitoyl-2-myristoyl-sn-glycero-3-phosphocholine (16:0-14:0 PC, PMPC), 1-palmitoyl-2-stearoyl-sn-glycero-3-phosphocholine (16:0-18:0 PC, PSPC), 1-palmitoyl-2-oleoyl-sn-glycero-3-phosphocholine (16:0-18:1 PC, POPC), 1-palmitoyl-2-linoleoyl-sn-glycero-3-phosphocholine (16:0-18:2 PC, PLPC), 1-palmitoyl-2-arachidonoyl-sn-glycero-3-phosphocholine (16:0-20:4 PC), 1-palmitoyl-2-docosahexaenoyl-sn-glycero-3-phosphocholine (14:0-22:6 PC), 1-stearoyl-2-myristoyl-sn-glycero-3-phosphocholine (18:0-14:0 PC, SMPC), 1-stearoyl-2-palmitoyl-sn-glycero-3-phosphocholine (18:0-16:0 PC, SPPC), 1-stearoyl-2-oleoyl-sn-glycero-3-phosphocholine (18:0-18:1 PC, SOPC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero phosphocholine (18:0-22:6 PC), 1-oleoyl-2-myristoyl-sn-glycero-3-phosphocholine (18:1-14:0 PC, OMPC), 1-oleoyl-2-palmitoyl-sn-glycero-3-phosphocholine (18:1-16:0 PC, OPPC), 1-oleoyl-2-stearoyl-sn-glycero-3-phosphocholine (18:1-18:0 PC, OSPC), 1-palmitoyl oleoyl-sn-glycero-3-phosphoethanolamine (16:0-18:1 PE, POPE), 1-palmitoyl-2-linoleoyl-sn-glycero-3-phosphoethanolamine (16:0-18:2 PE), 1-palmitoyl-2-arachidonoyl-sn-glycero phosphoethanolamine (16:0-20:4 PE), 1-palmitoyl-2-docosahexaenoyl-sn-glycero phosphoethanolamine (16:0-22:6 PE), 1-stearoyl-2-oleoyl-sn-glycero-3-phosphoethanolamine (18:0-18:1 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-cholesterylhemisuccinoyl-sn-glycero-3-phosphocholine (OChemsPC), and any combination thereof.

Asymmetric lipids useful in the lipid composition may also be lysolipids. Lysolipids may be selected from the non-limiting group consisting of 1-hexanoyl-2-hydroxy-sn-glycero-3-phosphocholine (06:0 Lyso PC), 1-heptanoyl-2-hydroxy-sn-glycero-3-phosphocholine (07:0 Lyso PC), 1-octanoyl-2-hydroxy-sn-glycero-3-phosphocholine (08:0 Lyso PC), 1-nonanoyl-2-hydroxy-sn-glycero-3-phosphocholine (09:0 Lyso PC), 1-decanoyl-2-hydroxy-sn-glycero-3-phosphocholine (10:0 Lyso PC), 1-undecanoyl-2-hydroxy-sn-glycero-3-phosphocholine (11:0 Lyso PC), 1-lauroyl-2-hydroxy-sn-glycero-3-phosphocholine (12:0 Lyso PC), 1-tridecanoyl-2-hydroxy-sn-glycero-3-phosphocholine (13:0 Lyso PC), 1-myristoyl-2-hydroxy-sn-glycero-3-phosphocholine (14:0 Lyso PC), 1-pentadecanoyl-2-hydroxy-sn-glycero-3-phosphocholine (15:0 Lyso PC), 1-palmitoyl-2-hydroxy-sn-glycero-3-phosphocholine (16:0 Lyso PC), 1-heptadecanoyl-2-hydroxy-sn-glycero-3-phosphocholine (17:0 Lyso PC), 1-stearoyl-2-hydroxy-sn-glycero-3-phosphocholine (18:0 Lyso PC), 1-oleoyl-2-hydroxy-sn-glycero-3-phosphocholine (18:1 Lyso PC), 1-nonadecanoyl-2-hydroxy-sn-glycero-3-phosphocholine (19:0 Lyso PC), 1-arachidoyl-2-hydroxy-sn-glycero-3-phosphocholine (20:0 Lyso PC), 1-behenoyl-2-hydroxy-sn-glycero-3-phosphocholine (22:0 Lyso PC), 1-lignoceroyl-2-hydroxy-sn-glycero-3-phosphocholine (24:0 Lyso PC), 1-hexacosanoyl-2-hydroxy-sn-glycero-3-phosphocholine (26:0 Lyso PC), 1-myristoyl-2-hydroxy-sn-glycero-3-phosphoethanolamine (14:0 Lyso PE), 1-palmitoyl-2-hydroxy-sn-glycero-3-phosphoethanolamine (16:0 Lyso PE), 1-stearoyl-2-hydroxy-sn-glycero-3-phosphoethanolamine (18:0 Lyso PE), 1-oleoyl-2-hydroxy-sn-glycero-3-phosphoethanolamine (18:1 Lyso PE), 1-hexadecyl-sn-glycero-3-phosphocholine (C16 Lyso PC), and any combination thereof.

In some aspects, the lipid composition of a nanoparticle disclosed herein comprises at least one asymmetric phospholipid selected from the group consisting of sphingosine, MPPC, MSPC, PMPC, PSPC, SMPC, SPPC, and any combination thereof. In some aspects, the asymmetric phospholipid is sphingosine.

In some aspects, the lipid composition of a NP disclosed herein comprises at least one symmetric phospholipid. Symmetric phospholipids may be selected from the non-limiting group consisting of 1,2-dipropionyl-sn-glycero-3-phosphocholine (03:0 PC), 1,2-dibutyryl-sn-glycero-3-phosphocholine (04:0 PC), 1,2-dipentanoyl-sn-glycero-3-phosphocholine (05:0 PC), 1,2-dihexanoyl-sn-glycero-3-phosphocholine (06:0 PC), 1,2-diheptanoyl-sn-glycero-3-phosphocholine (07:0 PC), 1,2-dioctanoyl-sn-glycero-3-phosphocholine (08:0 PC), 1,2-dinonanoyl-sn-glycero-3-phosphocholine (09:0 PC), 1,2-didecanoyl-sn-glycero-3-phosphocholine (10:0 PC), 1,2-diundecanoyl-sn-glycero-3-phosphocholine (11:0 PC, DUPC), 1,2-dilauroyl-sn-glycero-3-phosphocholine (12:0 PC), 1,2-ditridecanoyl-sn-glycero-3-phosphocholine (13:0 PC), 1,2-dimyristoyl-sn-glycero-3-phosphocholine (14:0 PC, DMPC), 1,2-dipentadecanoyl-sn-glycero-3-phosphocholine (15:0 PC), 1,2-dipalmitoyl-sn-glycero-3-phosphocholine (16:0 PC, DPPC), 1,2-diphytanoyl-sn-glycero-3-phosphocholine (4ME 16:0 PC), 1,2-diheptadecanoyl-sn-glycero-3-phosphocholine (17:0 PC), 1,2-distearoyl-sn-glycero-3-phosphocholine (18:0 PC, DSPC), 1,2-dinonadecanoyl-sn-glycero-3-phosphocholine (19:0 PC), 1,2-diarachidoyl-sn-glycero-3-phosphocholine (20:0 PC), 1,2-dihenarachidoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-dimyristoleoyl-sn-glycero-3-phosphocholine (14:1 (A9-Cis) PC), 1,2-dimyristelaidoyl-sn-glycero-3-phosphocholine (14:1 (A9-Trans) PC), 1,2-dipalmitoleoyl-sn-glycero-3-phosphocholine (16:1 (A9-Cis) PC), 1,2-dipalmitelaidoyl-sn-glycero-3-phosphocholine (16:1 (A9-Trans) PC), 1,2-dipetroselenoyl-sn-glycero-3-phosphocholine (18:1 (A6-Cis) PC), 1,2-dioleoyl-sn-glycero-3-phosphocholine (18:1 (A9-Cis) PC, DOPC), 1,2-dielaidoyl-sn-glycero-3-phosphocholine (18:1 (A9-Trans) PC), 1,2-dilinoleoyl-sn-glycero-3-phosphocholine (18:2 (Cis) PC, DLPC), 1,2-dilinolenoyl-sn-glycero-3-phosphocholine (18:3 (Cis) PC, DLnPC), 1,2-dieicosenoyl-sn-glycero-3-phosphocholine (20:1 (Cis) PC), 1,2-diarachidonoyl-sn-glycero-3-phosphocholine (20:4 (Cis) PC, DAPC), 1,2-dierucoyl-sn-glycero-3-phosphocholine (22:1 (Cis) PC), 1,2-didocosahexaenoyl-sn-glycero-3-phosphocholine (22:6 (Cis) PC, DHAPC), 1,2-dinervonoyl-sn-glycero-3-phosphocholine (24:1 (Cis) PC), 1,2-dihexanoyl-sn-glycero-3-phosphoethanolamine (06:0 PE), 1,2-dioctanoyl-sn-glycero phosphoethanolamine (08:0 PE), 1,2-didecanoyl-sn-glycero-3-phosphoethanolamine (10:0 PE), 1,2-dilauroyl-sn-glycero-3-phosphoethanolamine (12:0 PE), 1,2-dimyristoyl-sn-glycero-3-phosphoethanolamine (14:0 PE), 1,2-dipentadecanoyl-sn-glycero-3-phosphoethanolamine (15:0 PE), 1,2-dipalmitoyl-sn-glycero-3-phosphoethanolamine (16:0 PE), 1,2-diphytanoyl-sn-glycero-3-phosphoethanolamine (4ME 16:0 PE), 1,2-diheptadecanoyl-sn-glycero phosphoethanolamine (17:0 PE), 1,2-distearoyl-sn-glycero-3-phosphoethanolamine (18:0 PE, DSPE), 1,2-dipalmitoleoyl-sn-glycero-3-phosphoethanolamine (16:1 PE), 1,2-dioleoyl-sn-glycero-3-phosphoethanolamine (18:1 (A9-Cis) PE, DOPE), 1,2-dielaidoyl-sn-glycero-3-phosphoethanolamine (18:1 (A9-Trans) PE), 1,2-dilinoleoyl-sn-glycero-3-phosphoethanolamine (18:2 PE, DLPE), 1,2-dilinolenoyl-sn-glycero-3-phosphoethanolamine (18:3 PE, DLnPE), 1,2-diarachidonoyl-sn-glycero-3-phosphoethanolamine (20:4 PE, DAPE), 1,2-didocosahexaenoyl-sn-glycero-3-phosphoethanolamine (22:6 PE, DHAPE), 1,2-di-O-octadecenyl-sn-glycero-3-phosphocholine (18:0 Diether PC), 1,2-dioleoyl-sn-glycero-3-phospho-rac-(1-glycerol) sodium salt (DOPG), and any combination thereof.

In some aspects, the lipid composition of a nanoparticle disclosed herein comprises at least one symmetric phospholipid selected from the non-limiting group consisting of DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof.

In some aspects, the lipid compositions disclosed herein may contain one or more symmetric phospholipids, one or more asymmetric phospholipids, or a combination thereof. When multiple phospholipids are present, they can be present in equimolar ratios, or non-equimolar ratios.

In one aspect, the lipid composition of a nanoparticle disclosed herein comprises a total amount of phospholipid (e.g., sphingolipid/DAPC) which ranges from about 30 mol % to about 50 mol %, from about 25 mol % to about 60 mol %, from about 40 mol % to about 80 mol %, from about 50 mol % to about 80 mol %, from about 30 mol % to about 90 mol %, in the lipid composition. In one aspect, the amount of the phospholipid is from about 8 mol % to about 15 mol % in the lipid composition. In one aspect, the amount of the phospholipid (e.g., MSPC) is about 10 mol % in the lipid composition.

In some aspects, the amount of a specific sphingosine is at least about 1, 5, 10, 20, or 30 mol % in the lipid composition. In some aspects, the amount of specific DAPC is at least about 1, 5, 10, 20, 30, 40, or 50 mol % in the lipid composition.

### B. Structural Lipids

The lipid composition of a nanoparticle disclosed herein can comprise one or more structural lipids. As used herein, the term “structural lipid” refers to sterols and also to lipids containing sterol moieties. In some aspects, the structural lipid is selected from the group consisting of cholesterol, fecosterol, sitosterol, ergosterol, campesterol, stigmasterol, brassicasterol, tomatidine, tomatine, ursolic acid, alpha-tocopherol, and mixtures thereof. In some aspects, the structural lipid is cholesterol.

In one aspect, the amount of the structural lipid (e.g., a sterol such as cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one aspect, the amount of the structural lipid (e.g., a sterol such as cholesterol) in the lipid composition disclosed herein ranges from about 10 mol % to about 25 mol %, or from about 15 mol % to about 20 mol %,

In some aspects, the amount of the structural lipid (e.g., a sterol such as cholesterol) in the lipid composition disclosed herein is at least about 10, 20, 25, 30, 35, 40, 45, 50, 55, or 60 mol %.

In some aspects, the lipid composition component of the pharmaceutical compositions for delivery disclosed does not comprise cholesterol.

### C. Polyethylene Glycol (PEG)-Lipids

The lipid composition of a nanoparticle disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC16), PEG-modified dialkylamines and PEG-modified 1,2-diacyloxypropan-3-amines Such lipids are also referred to as PEGylated lipids. For example, a PEG lipid may be PEG-c-DOMG, PEG-DMG, PEG-DLPE, PEG-DMPE, PEG-DPPC, or a PEG-DSPE lipid.

In some aspects, the PEG-lipid includes, but not limited to 1,2-dimyristoyl-sn-glycerol methoxypolyethylene glycol (PEG-DMG), 1,2-distearoyl-sn-glycero phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one aspect, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some aspects, the lipid moiety of the PEG-lipids includes those having lengths of from about C14 to about C22, preferably from about C14 to about C16. In some aspects, a PEG moiety has a size of about 1000, 2000, 5000, 10,000, 15,000 or 20,000 daltons.

In one aspect, the lipid nanoparticles described herein may comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO2015/130584, which are incorporated herein by reference in their entirety.

In one aspect, the amount of PEG-lipid in the lipid composition of a pharmaceutical composition disclosed herein ranges from about 0.1 mol % to about 10 mol %, from about 1 mol % to about 9 mol %, from about 2 mol % to about 8 mol %, from about 2 mol % to about 7 mol %, from about 3 mol % to about 6 mol %, or from about 4 mol % to about 5 mol %.

In one aspect, the amount of PEG-lipid in the lipid composition disclosed herein is about 5 mol %.

In one aspect, the amount of PEG-lipid in the lipid composition disclosed herein is at least about, 1, 2, 3, 3, 4, or 5, 10 mol %.

In some aspects, the lipid composition of the pharmaceutical compositions disclosed herein does not comprise a PEG-lipid.

In some aspects, the ratio of PEG in the nanoparticle (NP) formulations is increased or decreased and/or the carbon chain length of the PEG lipid is modified from C14 to C18 to alter the pharmacokinetics and/or biodistribution of the NP formulations.

### D. Quaternary Amine Compounds

The lipid composition of a nanoparticle disclosed herein can comprise one or more quaternary amine compounds (e.g., DOTAP). The term “quaternary amine compound” is used to include those compounds having one or more quaternary amine groups (e.g., trialkylamino groups) and permanently carrying a positive charge and existing in a form of a salt. For example, the one or more quaternary amine groups can be present in a lipid or a polymer (e.g., PEG). In some aspects, the quaternary amine compound comprises (1) a quaternary amine group and (2) at least one hydrophobic tail group comprising (i) a hydrocarbon chain, linear or branched, and saturated or unsaturated, and (ii) optionally an ether, ester, carbonyl, or ketal linkage between the quaternary amine group and the hydrocarbon chain. In some aspects, the quaternary amine group can be a trimethylammonium group. In some aspects, the quaternary amine compound comprises two identical hydrocarbon chains. In some aspects, the quaternary amine compound comprises two different hydrocarbon chains.

In some aspects, the lipid composition of a pharmaceutical composition disclosed herein comprises at least one quaternary amine compound. Quaternary amine compound may be selected from the non-limiting group consisting of 1,2-dioleoyl-3-trimethylammonium-propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-pr-opanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP) 1,2-distearoyl-sn-glycero-3-ethylphosphocholine (DSePC) 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof.

In one aspect, the quaternary amine compound is 1,2-dioleoyl trimethylammonium-propane (DOTAP).

Quaternary amine compounds are known in the art, such as those described in U.S. Patent Appl. Publ. Nos. US2013/0245107 and US2014/0363493, U.S. Pat. No. 8,158,601, and Intl. Publ. Nos. WO2015/123264 and WO2015/148247, which are incorporated herein by reference in their entireties.

In one aspect, the amount of the quaternary amine compound (e.g., DOTAP) in the lipid composition disclosed herein ranges from about 0.01 mol % to about 20 mol %.

In one aspect, the amount of the quaternary amine compound (e.g., DOTAP) in the lipid composition disclosed herein ranges from about 0.5 mol % to about 20 mol %, from about 1 mol % to about 10 mol %, or from about 4 mol % to about to about 15 mol %,

In one aspect, the amount of the quaternary amine compound (e.g., DOTAP) in the lipid composition disclosed herein ranges from about 5 mol % to about 10 mol %.

In one aspect, the amount of the quaternary amine compound (e.g., DOTAP) in the lipid composition disclosed herein is about 5 mol %. In one aspect, the amount of the quaternary amine compound (e.g., DOTAP) in the lipid composition disclosed herein is about 10 mol %.

In some aspects, the amount of the quaternary amine compound (e.g., DOTAP) is at least about 0.01, 0.1, 1, 2, 4, 6, 8, 10, 12, 14, 16, 18, or 20 mol % in the lipid composition disclosed herein.

In some aspects, the lipid composition of the pharmaceutical compositions disclosed herein does not comprise a quaternary amine compound. In some aspects, the lipid composition of the pharmaceutical compositions disclosed herein does not comprise DOTAP.

### E. Other Ionizable Amino Lipids

Ionizable lipids include 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3.beta.)-cholest-5-en-3-yloxy]octyl}oxy)-N,N-dimethyl-3-[(9Z,12Z)-octadeca-9,12-dien yloxy]propan-1-amine (Octyl-CLinDMA), (2R)-2-({8-[(3.beta.)-cholest-5-en yloxy]octyl}oxy)-N,N-dimethyl-3-[(9Z-,12Z)-octadeca-9,12-dien-1-yloxy]propan-1-amine (Octyl-CLinDMA (2R)), and (2S)-2-({8-[(3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid may also be a lipid including a cyclic amine group.

The lipid composition of a nanoparticle disclosed herein may include one or more components in addition to those described above. For example, the lipid composition may include one or more permeability enhancer molecules, carbohydrates, polymers, surface altering agents (e.g., surfactants), or other components. Carbohydrates may include simple sugars (e.g., glucose) and polysaccharides (e.g., glycogen and derivatives and analogs thereof). The lipid composition may include a buffer such as, but not limited to, citrate or phosphate at a pH of 7, salt and/or sugar. Salt and/or sugar may be included in the formulations described herein for isotonicity.

The ratio between the lipid composition and the nucleic acid in the mixture can range from about 10:1 to about 60:1 (wt/wt). In one aspect, the lipid nanoparticles/nucleic acid mixture described herein may comprise nucleic acid in a lipid:nucleic acid weight ratio of 5:1, 20:1, 50:1, or 70:1. In one aspect, the lipid nanoparticles/nucleic acid mixture described herein may comprise the nucleic acid in a concentration from approximately 0.1 mg/ml to 2 mg/ml.

A polymer may be included in and/or used to encapsulate or partially encapsulate the lipid nanoparticle/nucleic acid mixture. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

In some aspects, the lipoplex can comprise lipidoids. Lipidoids are lipid-like structures containing multiple secondary and tertiary amine functionalities which confer highly efficient interaction with anionic nucleic acid molecules. Lipidoids have been formulated as long-circulating stable nucleic acid lipid particles (SNALPs), also containing cholesterol and PEGylated phospholipids. Lipidoid-polymer hybrid nanoparticles (LPNs) are composed of lipidoids and poly(DL-lactic-co-glycolic acid) (PLGA). The synthesis of lipidoids has been extensively described and formulations containing these compounds are particularly suited for delivery of oligonucleotides or nucleic acids. Complexes, micelles, liposomes or particles can be prepared containing these lipidoids and therefore, can result in an effective delivery of a lipoplex mixture following the injection of a lipidoid formulation via localized and/or systemic routes of administration. Lipidoid-nucleic acid complexes can be administered by various means including, but not limited to, intravenous, intramuscular, or subcutaneous routes. The lipidoid formulations can include particles comprising other components in addition to the nucleic acid of the invention. As an example, formulations with certain lipidoids, include, but are not limited to, lipidoids that may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In some aspects the lipoplex can include dendrimers. Dendrimers are highly branched macromolecules. In one embodiment, a nucleic acid of the present invention is complexed with structurally flexible poly(amidoamine) (PAMAM) dendrimers. The complex is called nucleic acid-dendrimers. Dendrimers have a high degree of molecular uniformity, narrow molecular weight distribution, specific size and shape characteristics, and a highly-functionalized terminal surface. The manufacturing process is a series of repetitive steps starting with a central initiator core. Each subsequent growth step represents a new generation of polymers with a larger molecular diameter and molecular weight, and more reactive surface sites than the preceding generation.

PAMAM dendrimers are efficient nucleotide delivery systems that bear primary amine groups on their surface and also a tertiary amine group inside of the structure. The primary amine group participates in nucleotide binding and promotes their cellular uptake, while the buried tertiary amino groups act as a proton sponge in endosomes and enhance the release of nucleic acid into the cytoplasm. These dendrimers protect the nucleic carried by them from ribonuclease degradation and achieves substantial release of nucleic acid over an extended period of time via endocytosis for efficient gene targeting. The in vivo efficacy of these nanoparticles has previously been evaluated where biodistribution studies show that the dendrimers preferentially accumulate in peripheral blood mononuclear cells and live with no discernible toxicity (see Zhou et al., Molecular Ther. 2011 Vol. 19, 2228-2238, the contents of which are incorporated herein by reference in their entirety). PAMAM dendrimers may comprise a triethanolamine (TEA) core, a diaminobutane (DAB) core, a cystamine core, a diaminohexane (HEX) core, a diamonododecane (DODE) core, or an ethylenediamine (EDA) core. In one embodiment, PAMAM dendrimers comprise a TEA core or a DAB core.

In some aspects, the lipoplex can comprise a virus-like particle (VLP) or VLP nanoparticle (NP). VLP NPs are structurally and visually similar to live viruses but lack a complete virus genome or lack the entire virus genome and are noninfectious. VLPs are formed of self-assembling between one or more viral structural capsid proteins which can produce VLPs with geometrical symmetry, usually in the form of isosahedral, spherical, or rod-like structures, depending on from which virus the proteins are derived. The repetitive protein structure of VLPs can stimulate innate and adaptive immune systems. Some VLPs, such as those derived from HIV-1 and influenza virus, are enveloped VLPs and have a layer of lipid that contains viral surface antigens surrounding the capsid structure, reflecting the lipid envelope found in the natural infectious virus particle. Enveloped VLPs obtain their lipid membrane from the cell in which they are expressed during the assembly and budding of VLPs from the cell. The precise nature, origin and composition of the envelope are different for various enveloped VLPs and depends on the virus from which the VLP is derived which in turn determines the process of assembling and budding of the VLP from the host cell line that is used for their production. Methods for production (expression platforms including bacteria, baculovirus, plant cells, yeast, avian, mammalian cells, and cell free systems) and purification of VLPs and VLP NPs (clarification, depth filtration, microfiltration and tangential flow filtration, chromatography) are known in the art as well as analytical techniques for characterization of VLPs and VLP NPs. Biochemical methods include Matrix assisted laser desorption ionization time of flight-mass spectrometry (MALDI-TOF MS), liquid chromatography-mass spectrometry (LC-MS), sodium dodecyl sulphate polyacrylamide gel electrophoresis (SDS-PAGE) and reverse phase-high performance liquid chromatography (RP-HPLC). Biophysical methods include Transmission electron microscopy (TEM), cryo-electron microscopy (Cryo-EM), atomic force microscopy (AFM), asymmetric flow field-flow fractionation coupled with multiple-angle light scattering (AF4-MALS), electrospray differential mobility analysis (ES-DMA) and high performance size exclusion chromatography (HPSEC), dynamic light scattering (DLS), analytical ultracentrifugation (AUC), Circular dichroism (CD), differential scanning calorimetry (DSC), Cloud point. Biological characterization methods include Surface plasmon resonance (SRP), enzyme-linked immunosorbent assay (ELISA). Design of VLP NPs can include scaffolds such as Ferritin or encapsuling. NPs can be designed with specific antigens exposed on the surface for stimulating specific cells in the immune system or for targeting to a specific tissue.

Nanoparticles may include one or more lipids, may be in the form of a liposome, may include a lipid monolayer or bilayer, or be formed of micelles. In some embodiments, nanoparticles include a polymeric core surrounded by a lipid layer (e.g., lipid bilayer, lipid monolayer, etc.). In some embodiments, a nanoparticle includes a non-polymeric core (e.g., metal particle, quantum dot, ceramic particle, bone particle, etc.) surrounded by a lipid layer (e.g., lipid bilayer, lipid monolayer, etc.).

The nanoparticles are typically formed using an emulsion process, single or double, using an aqueous and a non-aqueous solvent or self-assembly of amphiphilic polymers or a mixture of amphiphlic polymers and hydrophobic polymers. Typically, the nanoparticles contain a minimal amount of the non-aqueous solvent after solvent removal. Preferred methods of preparing these nanoparticles are described in the examples.

In one embodiment, nanoparticles are prepared using emulsion solvent evaporation method. A polymeric material is dissolved in a water immiscible organic solvent and mixed with a drug solution or a combination of drug solutions. The water immiscible organic solvent is preferably a GRAS ingredient such as chloroform, dichloromethane, and acyl acetate. The drug can be dissolved in, but is not limited to, one or a plurality of the following: acetone, ethanol, methanol, isopropyl alcohol, acetonitrile and Dimethyl sulfoxide (DMSO). An aqueous solution is then added into the resulting mixture solution to yield emulsion solution by emulsification. The emulsification technique can be, but not limited to, probe sonication or homogenization through a homogenizer.

In another embodiment, nanoparticles are prepared using nanoprecipitation methods or microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution. The agents may be associated with the surface of, encapsulated within, surrounded by, and/or distributed throughout the polymeric matrix of the particles.

In another embodiment, nanoparticles are prepared by the self-assembly of the amphiphilic polymers, optionally including hydrophilic and/or hydrophobic polymers, using emulsion solvent evaporation, a single-step nanoprecipitation method, or microfluidic devices.

In one aspect, the nucleic acids are formulated in a swellable nanoparticle. In one aspect, the nanoparticles and microparticles of the present disclosure can be geometrically engineered to modulate macrophage and/or the immune response. In one aspect, the geometrically engineered particles can have varied shapes, sizes and/or surface charges. Other physical features the geometrically engineering particles can include, but are not limited to, fenestrations, angled arms, asymmetry and surface roughness, charge which can alter the interactions with cells and tissues.

In some aspects, the lipid nanoparticles have a diameter from about 10 to about 100 nm. In one embodiment, the nanoparticles have a diameter from about 10 to 500 nm. In one embodiment, the nanoparticle has a diameter greater than 500 nm, greater than 700 nm, greater than 900 nm, greater than 950 nm or greater than 1000 nm.

A nanoparticle composition may be relatively homogenous. A polydispersity index may be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition may have a polydispersity index from about 0 to about 0.25, such as 0-0.10. In some embodiments, the polydispersity index of a nanoparticle composition disclosed herein may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition disclosed herein may be from about −10 mV to about +20 mV.

Nanoparticle Mimics. The nucleic acids can be encapsulated within and/or absorbed to a nanoparticle mimic A nanoparticle mimic can mimic the delivery function organisms or particles such as, but not limited to, pathogens, viruses, bacteria, fungus, parasites, prions and cells. As a non-limiting example, the polynucleotides of the disclosure can be encapsulated in a non-virion particle which can mimic the delivery function of a virus. Nanoparticle mimics can include virus like particles.

Nanotubes. The polynucleotide nucleic acids can be attached or otherwise bound to at least one nanotube such as, but not limited to, rosette nanotubes, rosette nanotubes having twin bases with a linker, carbon nanotubes and/or single-walled carbon nanotubes. The polynucleotides can be bound to the nanotubes through forces such as, but not limited to, steric, ionic, covalent and/or other forces.

Microparticles. In some aspects, formulations comprising a nucleic acid can comprise microparticles. The microparticles can comprise a polymer described herein and/or known in the art such as, but not limited to, poly(α-hydroxy acid), a polyhydroxy butyric acid, a polycaprolactone, a polyorthoester and a polyanhydride. The microparticle can have adsorbent surfaces to adsorb biologically active molecules such as polynucleotides.

As used herein, the lipoplex composition comprises a nucleic acid. The nucleic acid can include synthetic and natural nucleic acids (including RNA, DNA, anti-sense RNA, triplex DNA, inhibitory RNA (RNAi), short interfering RNA (siRNA), short double-stranded hairpin-like RNA (shRNA), micro RNA (miRNA), threose nucleic acid (TNA), glycol nucleic acid (GNA), peptide nucleic acid (PNA), locked nucleic acid (LNA), and oligonucleotides), and biologically active portions thereof. Nucleic acids with lengths above about 10 bp are typically used. More typically, useful lengths of nucleic acids will be in the range from about 20 bp to tens of thousands of bp, including genes and vectors. Nucleic acid analogs can be modified at the base moiety, sugar moiety, or phosphate backbone. Such modification can improve, for example, stability, hybridization, solubility, immunogenicity, or targeting of the nucleic acid. Exemplary modifications include 2′O-methyl, 2′ methoxyethyl, phosphoramidate, methylphosphonate, and/or phosphorothioate backbone chemistry. In some embodiments, nucleic acids are modified to acquire one or more properties selected from the group consisting of increase nuclease resistance, enhanced membrane permeability, and reduced immunogenicity

The chemical modifications include chemical modification of nucleobases, sugar moieties, nucleotide linkages, or combinations thereof. As used herein “modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone. Chemical modifications of heterocyclic bases or heterocyclic base analogs may be effective to increase the binding affinity or stability in binding a target sequence. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino (2′-deoxy-.beta.-D-ribofuranosyl)pyridine (2-aminopyridine), and various pyrrolo- and pyrazolopyrimidine derivatives.

Sugar moiety modifications include, but are not limited to, 2′-O-aminoetoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA).

In some embodiments, the functional nucleic acid is a morpholino oligonucleotide. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tm, even with oligomers as short as 10-14 bases; the ability of the oligomer to be actively transported into mammalian cells; and the ability of an oligomer:RNA heteroduplex to resist RNAse degradation.

Modifications to the phosphate backbone of DNA or RNA oligonucleotides may increase the binding affinity or stability oligonucleotides, reduce the susceptibility of oligonucleotides nuclease digestion, or increase membrane permeability. Cationic modifications, including, but not limited to, diethyl-ethylenediamide (DEED) or dimethyl-aminopropylamine (DMAP) may be especially useful due to decrease electrostatic repulsion between the oligonucleotide and a target. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

The oligonucleotides can be locked nucleic acids. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

In some embodiments, the oligonucleotides are composed of peptide nucleic acids. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds. PNAs maintain spacing of heterocyclic bases that is similar to conventional DNA oligonucleotides but are achiral and neutrally charged molecules. Peptide nucleic acids are formed of peptide nucleic acid monomers.

Other backbone modifications include peptide and amino acid variations and modifications. Thus, the backbone constituents of oligonucleotides such as PNA may be peptide linkages, or alternatively, they may be non-peptide peptide linkages. Examples include acetyl caps, amino spacers such as 8-amino-3,6-dioxaoctanoic acid (referred to herein as 0-linkers), amino acids such as lysine are particularly useful if positive charges are desired in the PNA, and the like. Methods for the chemical assembly of PNAs are well known.

Oligonucleotides optionally include one or more terminal residues or modifications at either or both termini to increase stability, and/or affinity of the oligonucleotide for its target. Commonly used positively charged moieties include the amino acids lysine and arginine, although other positively charged moieties may also be useful. Oligonucleotides may further be modified to be end capped to prevent degradation using a propylamine group. Procedures for 3′ or 5′ capping oligonucleotides are well known in the art.

The nucleic acid molecule can exist as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, etc. The nucleic acid can be an engineered nucleic acid such as a recombinant DNA molecule that is part of a hybrid or fusion nucleic acid.

The nucleic acid in the NP/nucleic acid need only be associated with the nanoparticle composition after preparation. It can be partially, substantially or completely enclosed, surrounded or encased by the nanoparticle. Fluorescence may be used to measure the amount of free polynucleotide in a solution. The term “encapsulation efficiency” of a polynucleotide describes the amount of the polynucleotide that is encapsulated by a nanoparticle composition. For the compositions described herein, the encapsulation efficiency of a polynucleotide may be 10% or less, 20% or less, 50% or less.

The amount of a polynucleotide present in a lipoplex composition disclosed herein can depend on multiple factors, for example, the amount of a nucleic acid may depend on the size (expressed as length, or molecular mass), sequence, and other characteristics of the nucleic acid. The relative amounts of a polynucleotide in a nanoparticle composition may also vary.

In one aspect, such formulations are also constructed or compositions altered such that they passively or actively are directed to different cell types in vivo, including but not limited to hepatocytes, immune cells, tumor cells, endothelial cells, antigen presenting cells, and leukocytes. Formulations can also be selectively targeted through expression of different ligands on their surface as exemplified by, but not limited by, folate, transferrin, N-acetylgalactosamine (GalNAc), and antibody targeted approaches.

In another aspect, the lipid nanoparticle is encapsulated into any polymer known in the art which can form a gel when injected into a subject. As another non-limiting example, the lipid nanoparticle is encapsulated into a polymer matrix which can be biodegradable. In one aspect, the nanoparticles of the present disclosure comprise a polymeric matrix. As a non-limiting example, the nanoparticle comprises two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one aspect, the nanoparticle comprises a diblock or multi-block copolymer. In one embodiment, the diblock copolymer includes PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one aspect, the nanoparticle comprises at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one aspect, the nanoparticles comprise at least one poly(vinyl ester) polymer. The poly(vinyl ester) polymer can be a copolymer such as a random copolymer. In one aspect, the poly(vinyl ester) polymers can be conjugated to the polynucleotides described herein.

In one aspect, the nanoparticles comprise at least one degradable polyester which can contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer.

In another aspect, the nanoparticle includes a conjugation of at least one targeting ligand. The targeting ligand can be any ligand known in the art such as, but not limited to, a monoclonal antibody.

In one aspect, the synthetic nanocarriers contain an immunostimulatory agent to enhance the immune response from delivery of the synthetic nanocarrier. As a non-limiting example, the synthetic nanocarrier can comprise a Th1 immunostimulatory agent which can enhance a Th1-based response of the immune system.

In another aspect, the lipid particle described herein are formulated in nanoparticles used in imaging and can comprise lipids comprising an imaging agent such as gadolinium, e.g. gadolinium(III)2-{4,7-bis-carboxymethyl-10-[(N,N-distearylamidomethyl-N′-amido-methyl]-1,4,7,10-tetra-azacyclododec-1-yl}-acetic acid.

By ‘predosing’ or ‘pretreating’ is meant contacting a tissue with a tightening agent such as a lipoplex composition and/or an IFN-λ composition such that tightening of the epithelium in the tissue restricts entry of a drug or imaging agent into the tissue. Predosing can be an initial dose or multiple doses of a lipoplex composition. As used herein predosing means administering an effective amount of an IFN-λ composition and/or a nanoparticle mimic, e.g., a virus-like particle and a nucleic acid, or a lipoplex composition comprising a lipid nanoparticle (NP) and a nucleic acid prior to administering an anti-cancer drug or imaging agent, wherein the prior administration is effective to induce tightening of epithelial junctions in nontumor tissue. That is, the concentration, route of administration and timing of administration of the IFN-λ or nanoparticle mimic composition are effective to induce tightening of epithelial junctions in nontumor tissue.

Cell junctions fall into three functional classes: occluding junctions, anchoring junctions, and communicating junctions. Epithelial tight junctions are occluding junctions between epithelial cells that function as selective permeability barriers and are crucial in maintaining the concentration differences of small hydrophilic molecules across epithelial cell sheets forming the epithelium.

By ‘epithelium’ is meant a membranous body tissue consisting of epithelial cells closely packed together and joined by cell junctions, and not classified as connective, muscle, or neuronal. The epithelium may be classified based on the number of layers that make up the tissue: simple, stratified, and pseudostratified. It may also be classified histologically, by cell shape: squamous, columnar, and cuboidal. The epithelium forms the covering the external surfaces of the body (i.e., skin), and lines a tube or body cavity or organ, gland and other structures within the body.

By ‘endothelium’; is meant a specialized type of epithelial cell that lines the internal surfaces of the component of the circulatory system and consist of simple squamous epithelium.

Epithelial tight junction tightening is the ability of the epithelium to “tighten” to prevent access of agents, e.g., viruses and nanoparticles that are immobile, access to underlying tissues primarily via any active transport process such as a receptor-mediated process, or any passive transport process, such as the paracellular transport route. Paracellular transport refers to transport that occurs in between cells, passing through an intercellular shunt pathway. Normally, enhancing the epithelial tightening is an immune mechanism that is triggered in response to infection in an effort to reduce or limit further viral entry.

Epithelial tight junction tightening in the small intestines is the ability of the intestinal epithelial junctions formed between adjacent intestinal epithelia to tighten and create a physical intestinal barrier, to prevent access of agents, e.g., viruses and nanoparticles that are immobile, access to the intestinal lumen by regulating paracellular movement.

Methods for assessing endothelial and epithelial tightening are described below and known in the art. For example, transepithelial/transendothelial electrical resistance (TEER) is used to measure the integrity of tight junction dynamics TEER measurements can be performed in real-time without cell damage and are generally based on measuring ohmic resistance, or electrical resistance across a cellular monolayer, or measuring impedance across a spectrum of frequencies. TEER can be applied to monitor live cells during various stages. TEER measurement systems, such as Epithelial Voltohmmeter (EVOM) are commercially available. Flux of tracers (expressed as permeability coefficient) indicates the paracellular flow, as well as pore size of the tight junctions.

In another aspect, a method of treating cancer in a subject comprises predosing the subject with an effective amount of an IFN-λ composition, or an effective amount of a lipoplex composition, the lipoplex composition comprising a lipid nanoparticle (NP) and a nucleic acid, wherein the predosing with the effective amount is effective to induce tightening of epithelial junctions in nontumor tissue, and administering a therapeutically effective amount of an anti-cancer agent wherein predosing provides accumulation/uptake of the anti-cancer agent in tumor tissue compared to accumulation/uptake in nontumor tissue.

“Anti-cancer” agent includes physiologically or pharmacologically active substances that act locally or systemically in the body. An anti-cancer agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment. The agent can include therapeutic, nutritional, diagnostic, and prophylactic agents. The agents can be small molecule agents or biomacromolecules, such as proteins, polypeptides, sugars or carbohydrates, lipids, nucleic acids or small molecule compounds (typically 1 kD or less but may be larger). Suitable small molecule agents include organic and organometallic compounds. The small molecule agents can be a hydrophilic, hydrophobic, or amphiphilic compound.

Anti-cancer agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), and biologically active portions thereof. Suitable agents have a size greater than about 1,000 Da for small peptides and polypeptides, more typically at least about 5,000 Da and often 10,000 Da or more for proteins. Nucleic acids are more typically listed in terms of base pairs or bases (collectively “bp”). Nucleic acids with lengths above about 10 bp are typically used. More typically, useful lengths of nucleic acids for probing or therapeutic use will be in the range from about 20 bp (probes; inhibitory RNAs, etc.) to tens of thousands of bp for genes and vectors. The agents may also be hydrophilic molecules, preferably having a low molecular weight.

Exemplary therapeutic agents include cytokines, chemotherapeutic agents, radionuclides, monoclonal antibodies or other immunotherapeutics, enzymes, antivirals, anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, immunomodulators (including ligands that bind to Toll-Like Receptors (including, but not limited to, CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of particles into cells, and nutraceuticals such as vitamins

In another aspect, a method of imaging a cancer in a subject, comprising predosing the subject with an effective amount of an epithelial tightening agent such as an IFN-λ composition, an IFN-λ in combination with a virus-like particle with nucleic acid composition, or in combination with a lipoplex composition, or a VLP and nucleic acid composition, or a lipoplex composition, the lipoplex composition comprising a lipid nanoparticle (NP) and a nucleic acid, wherein the predosing with the effective amount is effective to induce tightening of epithelial junctions in nontumor tissue, and administering a imaging agent wherein predosing provides accumulation/uptake of the imaging agent in tumor tissue compared to accumulation/uptake in nontumor tissue.

Exemplary diagnostic agents include paramagnetic molecules, fluorescent compounds, magnetic molecules, and radionuclides, x-ray imaging agents, and contrast agents. An imaging, detectable or sensing moiety, i.e., a moiety that can be determined in some fashion, either directly or indirectly, may be bound to the NPs or to the polymers forming the NPs, or encapsulated therein. Representative imaging entities include, but are not limited to, fluorescent, radioactive, electron-dense, magnetic, or labeled members of a binding pair or a substrate for an enzymatic reaction, which can be detected. In some cases, the imaging entity itself is not directly determined, but instead interacts with a second entity in order to effect determination; for example, coupling of the second entity to the imaging entity may result in a determinable signal. Non-limiting examples of imaging moieties include, but are not limited to, fluorescent compounds such as FITC or a FITC derivative, fluorescein, green fluorescent protein (“GFP”), radioactive atoms such as ³H, ¹⁴C, ³³P, ³²P, ¹²⁵I, ¹³¹I, ³⁵S, or a heavy metal species, for example, gold or osmium. An imaging moiety may be a gold nanoparticle. A diagnostic or imaging tag such as a fluorescent tag can be chemically conjugated to a polymer to yield a fluorescently labeled polymer. For imaging, radioactive materials such as Technetium99 (99mTc) or magnetic materials such as Fe₂O₃ could be used. Examples of other materials include gases or gas emitting compounds, which are radioopaque.

### Cancers

The types of cancer that can be treated with the provided compositions and methods include, but are not limited to, the following: bladder, brain, breast, cervical, colorectal, esophageal, kidney, liver, lung, nasopharangeal, pancreatic, prostate, skin, stomach, uterine, ovarian, testicular and the like.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

Pharmaceutical compositions can be for administration by parenteral (intramuscular, intraperitoneal, intravenous (IV) or subcutaneous injection), routes of administration and can be formulated in dosage forms appropriate for each route of administration. The compositions are most typically administered systemically.

Compounds and pharmaceutical compositions thereof can be administered in an aqueous solution, by parenteral injection. The formulation may also be in the form of a suspension or emulsion, optionally including pharmaceutically acceptable diluents, preservatives, solubilizers, emulsifiers, adjuvants and/or carriers. Diluents include sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives. Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

Injectable preparations (e.g., intratumoral), for example, sterile injectable aqueous or oleaginous suspensions can be formulated according to the known art and can include suitable dispersing agents, wetting agents, and/or suspending agents. Sterile injectable preparations can be sterile injectable solutions, suspensions, and/or emulsions in nontoxic parenterally acceptable diluents and/or solvents, for example, a solution in 1,3-butanediol. Among the acceptable vehicles and solvents that can be employed include, but are not limited to, water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Any of the pharmaceutical compositions can be formulated for pulmonary or mucosal administration. The administration can include delivery of the composition to the lungs, nasal, oral (sublingual, buccal), vaginal, or rectal mucosa.

Suitable parenteral administration routes include intravascular administration; subcutaneous injection or deposition including subcutaneous infusion (such as by osmotic pumps); or direct application by a catheter or other placement device (e.g., an implant comprising a porous, non-porous, or gelatinous material).

The IFN-λ and/or nanoparticle mimic, e.g., VLP or lipoplex with a nucleic acid, formulation can be administered in a single dose or in multiple doses. Dosage levels on the order of about 1 mg/kg to 100 mg/kg of body weight per administration are useful in eliciting endothelial tightening. One skilled in the art can also readily determine an appropriate dosage regimen for administering any the disclosed IFN-λ, VLP, or lipoplex compositions. For example, the formulation can be administered to the subject once, e.g., as a single injection, infusion or bolus. Alternatively, the formulation can be administered once or twice daily to a subject for a period of from about three to about twenty-eight days, or from about seven to about ten days. A drug or imaging agent can be administered at any time prior to, during or after pre-dosing, at a time interval ranging from 3 minutes to 30 days or more, with additional intervening administration of predosing lipoplex composition as necessary to achieve or retain tightening. Predosing can be repeated prior to each dose of the anti-cancer agent, prior to every two doses, three doses, four doses, or five doses of the anti-cancer agent. Predosing can be repeated twice, three times four times, or five times prior to receiving a dose of the anti-cancer agent. Predosing can be repeated at about 10 minutes to about 24 hours. Predosing can be administered from about 5-120 minutes, or from about 3 hours to 28 days, prior to administration of the anti-cancer agent.

In another aspect, a method for selectively reducing, accumulation or uptake in nontumor tissue of a patient while enhancing delivery or deposition of a desired agent in a tumor comprising administering to said patient an effective amount of an IFN-λ composition, an effective amount of IFN-λ and lipoplex composition, or an effective amount of a lipoplex composition, the lipoplex composition comprising a lipid nanoparticle (NP) and a nucleic acid, wherein the effective amount is effective for inducing tightening of epithelial junctions in nontumor tissue, and reducing accumulation or uptake in nontumor tissue compared to delivery or deposition of the desired agent in a tumor.

The invention is further illustrated by the following non-limiting examples.

### Materials and Methods

The following Materials and Methods were used in the Examples below.

### Materials and Methods for Examples 1-4

### Materials

Cholesterol, diarachidoyl-sn-glycero-3-phosphocholine (DAPC), sphingosine, lactosylceramide (C-16), and PEG750-ceramide (C-16) were obtained from Avanti® Polar Lipids (Alabaster, Ala.).

### Lipoplex Preparation

Lipids were dissolved in chloroform and used to prepare liposomes comprised of sphingosine:cholesterol:DAPC at a mole ratio of 3:2:5 in water as previously described. Briefly, the lipid mixture was dried with nitrogen gas to achieve a thin film that was incubated under vacuum overnight before rehydration with distilled water and sonication to achieve clarity (1-2 min). Lactosylceramide or PEG-ceramide were included in the chloroform-lipid mixture and incorporated into liposomes at a 5% mole ratio as previously described. Lipoplexes were prepared by combining liposomes at an N/P ratio of 0.5 with an equal volume of an altered (CMV removed, ROSA26 added) pSelect-LucSh (InvivoGen, San Diego, Calif.) plasmid encoding luciferase (purified and eluted in water via the “salt-sensitive” protocol and stored at −20° C.). The diameters of these preparations were 237.9±7.5 (lactose) and 273.4±10.4 (PEG) nm as reported previously.

### Animal Studies

For in vivo experiments, lipoplex preparations were concentrated by centrifugation with a Centricon® filter as previously described, and they were then diluted 1:1 (v/v) with 12% hydroxyl ethyl starch (MW 250,000, Fresenius; Linz, Austria) immediately prior to intravenous administration.

Prior to treatment with lipoplexes, female immunocompetent Balb/c mice 6-8 weeks old were purchased from Jackson labs (Bar Harbor, Me.) and inoculated in the flank with 1×10⁶ CT26murine colon carcinoma (ATCC® CRL-2638). When tumor volumes reached approximately 80-100 mm³ (approximately 7 days), each mouse received an intravenous tail vein injection of lipoplexes, and the animals were sacrificed 24 or 72 h after the first or second injection. In an attempt to simulate clinical infusion, suspensions containing 50 μg plasmid in 200 μl were slowly injected (over approximately 10 seconds) via tail vein as previously described. We have demonstrated that this approach sharply reduces expression in tumors and organs (>100-fold) by avoiding hydrodynamic effects of a rapid bolus injection that are known to enhance delivery (data not shown). Luciferase expression was monitored in extracted tissues with Promega Luciferase Assay Reagents (Madison, Wis.) as previously described. All animal procedures were approved by the IACUC committee of The University of Colorado Anschutz Medical Campus and conformed to the guidelines established by the National Institutes of Health.

### Measurement of Plasmid Blood Levels

To monitor plasmid levels in the blood, mice were bled (15-50 μl) at 60 minutes using their submandibular veins as previously described. Whole blood was collected and centrifuged (2,000×g for 10 minutes) to separate plasma from the blood cell fraction, and each sample was prepared with a Qiagen DNeasy® Blood and Tissue kit (Qiagen, Germantown, Md.). Both the plasma and cell pellet were treated with the included lysis buffer per manufacturer's instructions. The samples were then subject to quantitative PCR using a QuantiTech® Reverse Transcription real-time RT-PCR Kit (Qiagen, Germantown, Md.) on an Applied Biosystems 7500 RTPCR instrument (Grand Island, N.Y.). A standard curve of pure plasmid was used to determine quantity. Although our PCR measurements do not directly determine whether the plasmid is associated with the lipoplex, previous studies have shown that free plasmid composed of naturally-occurring nucleotides is completely degraded (i.e., not detectable) within 10 min of intravenous administration in a mouse. While some studies have employed chemically-modified nucleotides that impart nuclease resistance, our study utilized unmodified DNA and thus we assume that the reported levels predominantly represent plasmid that is protected from degradation by its association with lipoplexes.

### Determination of Plasmid Levels in Tissues

To determine delivery of plasmid DNA to mouse tissues, animals were sacrificed 24 or 72 h after each lipoplex administration, and extracted organs were harvested and flash frozen in liquid nitrogen. Organs were subsequently thawed, weighed, and DNA was extracted using the Qiagen DNeasy® Blood and Tissue Kit (Qiagen, Germantown, Md.) as described above. Quantitative PCR (qPCR) was performed on tissue samples using QuantiTech® RTPCR Kit (Qiagen, Germantown, Md.) on an Applied Biosystems 7500 RTPCR instrument (Grand Island, N.Y.). A standard curve of pure plasmid was used for quantification in addition to amplicon efficiency factors that account for different efficiencies of amplification. We have previously determined the extraction efficiencies of plasmid from isolated organs, and these values were used to calculate the plasmid levels depicted in the results. It should be noted that the amplified PCR fragment is 1072 base pairs representing approximately one-sixth of the plasmid sequence. Therefore, our methods cannot distinguish between intact plasmid and large fragments that encode both primer sites. Previous studies have shown that free plasmid is rapidly degraded in tissues, and thus the presence of large fragments would be unlikely at the time points (24 and 72 h) used in our experiments.

### Cytokine Response

A separate set of tumor-bearing mice was used to quantify cytokine levels after repetitive injection of lipoplexes. In addition to the coated lipoplex formulations described above, mice were treated with phosphate buffered saline as a negative control. In these studies, triplicate mice were treated with lipoplex formulations as described above, and blood was collected 24 and 72 h after both the first and second injection of lipoplexes (3-day interval). Serum samples were allowed to clot for 30 minutes and spun at 2,000×g for 15 minutes per manufacturer's instructions (R&D Systems™, Minneapolis, Minn.). For solid tissues (liver, lung, spleen, tumor), extracted tissues were homogenized in buffer and cytokines extracted/quantified as described by the manufacturer. Samples were assayed in triplicate for interferon gamma (IFNγ), interleukin 6 (IL-6), and tumor necrosis factor alpha (TNFα) and using ELISA kits purchased from R&D Systems™ (Minneapolis Minn.). The limits of detection of IFNγ, IL-6, and TNFα are 2, 1, and 7 pg/mL for blood and 20, 40, 140 pg/g for tissues, respectively.

### Statistics

Significance was determined using a two-tailed unpaired t test and Taylor Series was used to calculate error for the in vitro PCR and luminescence measurements as different replicates were used including additional technical replicates on qPCR. All statistics and calculations were done with GraphPad Prism Software (San Diego, Calif.).

### Example 1

Many studies have documented the biodistribution and clearance of both synthetic (e.g., liposomes, polymeric nanoparticles, lipoplexes) and “natural” (e.g., viruses, exosomes) nanoparticles. The vast majority of this work has focused on applications for cancer treatment and therefore utilized tumor-bearing mouse models. As a method of improving cancer treatment, researchers have attempted to develop delivery systems that preferentially target tumors. In this regard, it is generally considered advantageous to design particles for prolonged circulation in order to maximize accumulation in the tumor. The predominant strategy for extending circulation times involves utilizing a polyethylene glycol (PEG) coating to prevent uptake by phagocytic cells involved in clearance. However, a significant drawback of utilizing PEGylated components is the development of anti-PEG antibodies that dramatically accelerate clearance of repeat intravenous doses of PEGylated nanoparticles. This phenomenon is known as “accelerated blood clearance” (ABC) and greatly limits the effectiveness of subsequent injections. These effects not only compromise delivery to tumors but can also elicit life-threatening immune reactions that have forced the termination of clinical trials. In fact, high levels of pre-existing anti-PEG antibodies have been correlated with adverse events during the infusion of therapeutic nanoparticles, and clinical trials in the U.S. are now required to monitor these antibodies before and after treatment. Accordingly, we and others have investigated alternative coatings that are less immunogenic.

Despite the predominant focus on the production of antibodies in response to nanoparticles, it is important to recognize that accelerated blood clearance is also observed in immunocompromised mice that are incapable of mounting an adaptive immune response. It follows that systemic innate immune responses must also contribute to the accelerated blood clearance phenomenon in addition to their role in the adverse infusion reactions noted above. In the context of evaluating the potential for adverse immune responses during infusion, studies have monitored the levels of blood cytokines at relatively early timepoints, i.e., 1-4 h. It is worth noting that the inclusion of nucleic acids appears to enhance the immunogenicity of PEG which further complicates the development of delivery vehicles for siRNA, mRNA, and genes. Our previous work has characterized the effects of different formulation variables (e.g., CpG, lipids, coatings) on the early cytokine response after repeated intravenous administration, and we have demonstrated that PEGylation elicits a potent, wide-ranging elevation of blood cytokines.

In addition to the heightened immunogenicity of PEG and the potential for systemic cytokine responses, it should also be recognized that mammalian cells have an extensive system for detecting the presence of foreign nucleic acids within the cytoplasm. This intricate detection system has evolved as a defense against viral infection and includes dozens of distinct mechanisms for preventing the expression of viral genes. Not surprisingly, this compromises the expression of plasmids containing a viral promoter (e.g., CMV), and it can also complicate the prolonged expression of therapeutic mRNA. Although these antiviral mechanisms are intracellular, infected cells produce cytokines that are secreted locally to inhibit viral replication in neighboring cells. It follows that cytokines involved in an antiviral response that limits gene expression are more localized and prolonged than those associated with a systemic infusion reaction. Therefore, the tissue-level antiviral response may not be accurately reflected by blood cytokine levels, especially when measured at early time points. Accordingly, we monitored both blood and tissue cytokines 24 and 72 hours after repeated intravenous administration of lipoplexes formulated with different coatings in an attempt to gain insight into the systemic and tissue-level immune responses elicited by lipoplexes and their effects on gene expression. Importantly, these experiments were conducted in a fully immunocompetent mouse strain bearing a murine colon tumor (CT26).

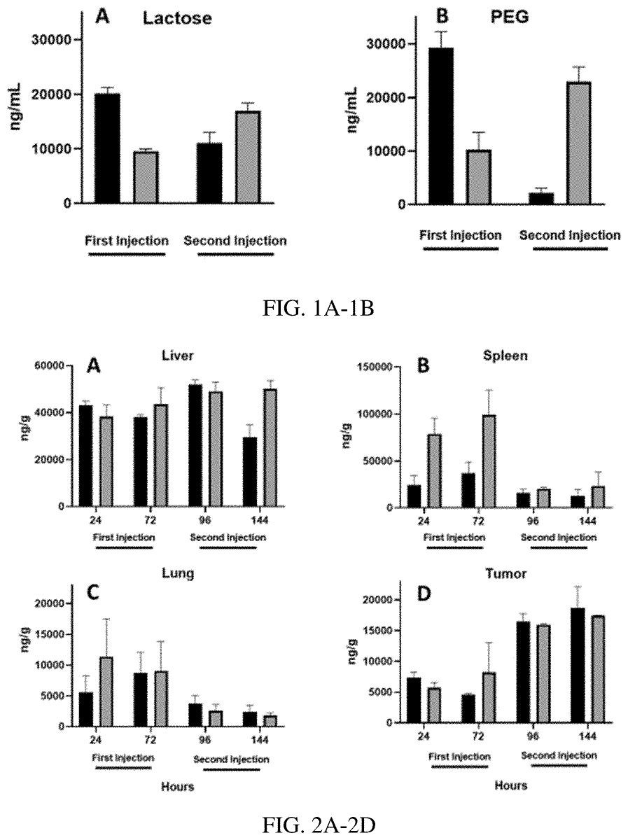

Lipoplexes were formulated with either 5% lactosylceramide or 5% PEG-ceramide and repeat administered (3-day interval) to tumor-bearing mice via tail vein injection. This dosing interval is consistent with our previous studies and is sufficient to avoid any potential saturation of uptake mechanisms that could affect repeat injections. To assess the role of coatings on accelerated blood clearance, blood was collected 1 h after each injection, and plasmid levels were assessed in both the plasma and cell fraction. As shown in FIG. 1, both coatings exhibited comparable levels (60-80% of injected dose) of plasmid in the blood (plasma+cell fractions) 1 h after the first injection, with the majority associated with the plasma. In contrast, both formulations were predominantly found in the cell fraction 1 h after the second injection with greater levels of plasmid from the lactose formulation in the plasma and higher amounts of PEGylated lipoplexes associated with blood cells. These results are consistent with our previous studies showing longer plasma circulation times provided by PEG as compared to lactose after a single injection and also with the accelerated clearance of PEGylated liposomes after repeat injections described in prior studies. However, we also observe accelerated plasma clearance of lactosylated lipoplexes after repeat injection (FIG. 1A). Taken together these results suggest that accelerated plasma clearance may be a more general phenomenon, at least for lipoplexes, consistent with the potent immune responses that are known to be elicited in response to nucleic acid-containing nanoparticles.

### Example 2

Previous studies have established that clearance by the liver, spleen, and lungs accounts for virtually all of the injected dose of lipoplexes after a single injection. In this study we monitored the accumulation of plasmid in these organs as well as the tumor 24 and 72 h after the first and second injections. After accounting for tissue weights, accumulation in the liver (≈1 g) was dramatically greater than other tissues (approximately 0.1 g) with significant accumulation also occurring in the spleen regardless of the coating (FIG. 2). Surprisingly, the second dose did not result in additional distribution to either of these organs or the lungs. In contrast to the absence of additional accumulation of the second dose in the liver, spleen, and lungs, deposition in the tumor approximately doubled after the second dose. Considering the accelerated clearance of the second dose, one might expect diminished accumulation. However, the absence of deposition after the second dose, especially in the liver and spleen, raises questions as to an alternative fate of repeat doses. The fact that the tumor exhibited supplemental accumulation after repeat dosing suggests that the accelerated blood clearance does not prevent additional deposition, consistent with our previous study demonstrating progressive increases in tumor accumulation after four doses of lipoplexes.

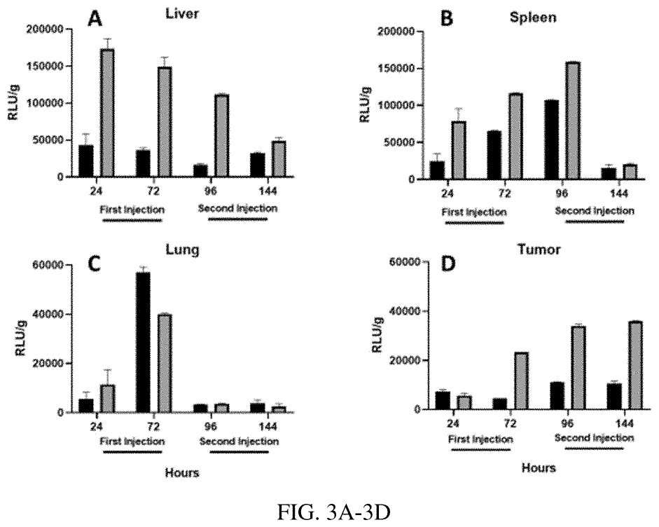

Changes in luciferase expression between the first and second injection generally corresponded with the observed changes in accumulation, i.e., expression in the liver, spleen, and lungs was not consistently enhanced after the second injection while that in the tumor maintained an approximately two-fold increase (FIG. 3). A closer look at the data reveals many cases where expression does not correlate with the amount of plasmid. For example, accumulation of plasmid in the liver was comparable for the two formulations (FIG. 2A), but expression was markedly higher for PEGylated lipoplexes at all time points (FIG. 3A). Similarly, luciferase expression in the spleen initially increased after the second injection despite sharp decreases in plasmid levels (compare FIGS. 2B+3B). Overall, the data indicate that expression is not well correlated with delivery, and that tissue type, coatings, timing, and repeat injection can each affect the levels of reporter gene expression that are observed in delivery studies.

### Example 3

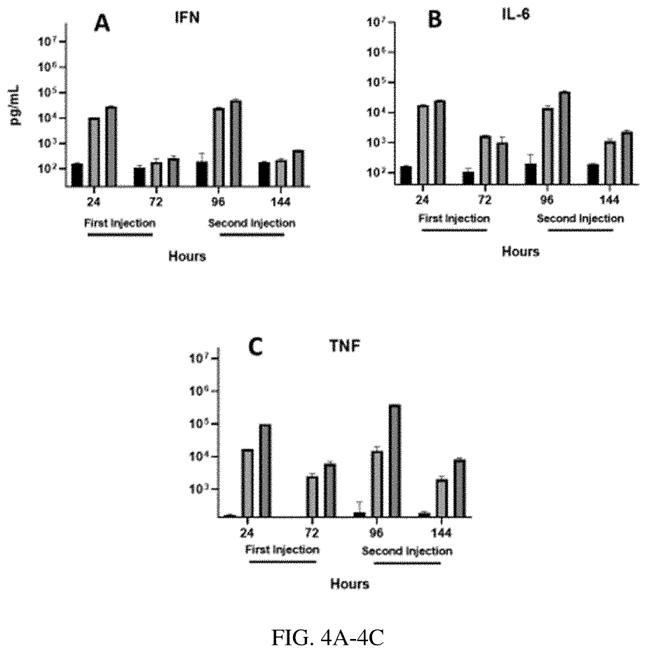

Cytokine levels in the blood are often used as a measure of the innate immune response, and excessive cytokines (“storm”) can be problematic. As mentioned above, cytokines are also secreted in response to microbial invasion and can silence gene expression. To evaluate the effect of lactose and PEG coatings on the cytokine response after repeat injection, we monitored blood levels of IFNγ, IL-6, and TNFα at 24 and 72 h after each injection. As shown in FIG. 4, each cytokine was elevated 24 h after the initial injection of lipoplexes and decreased to lower but still elevated, levels after 72 h. The second injection of lipoplexes elicited an increase in blood cytokines comparable to that seen 24 h after the first injection and again levels subsided after 72 h. In most cases, cytokines were more elevated after administration of PEGylated lipoplexes as compared to those coated with lactose, consistent with our previous findings.

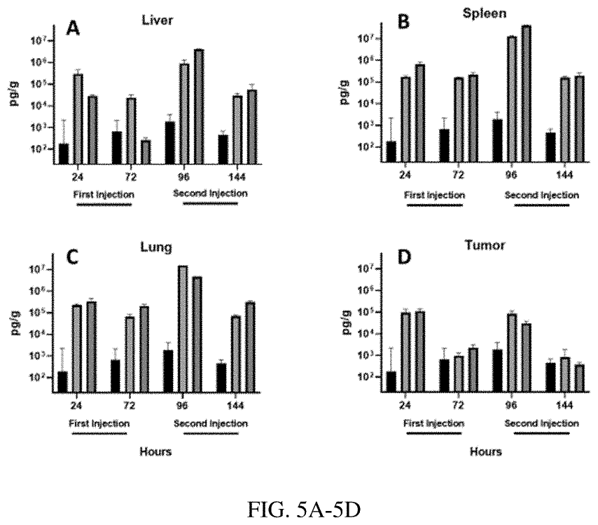

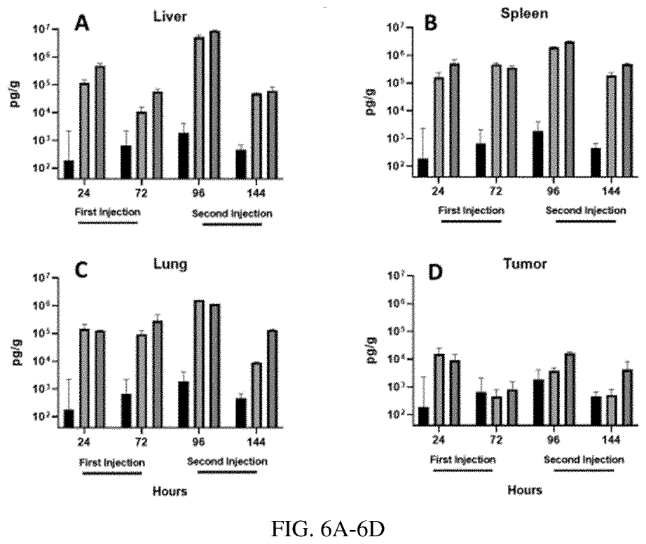

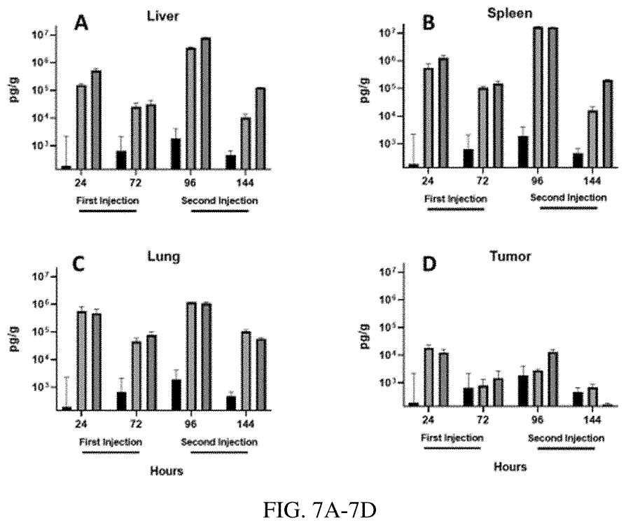

As compared to changes observed in the blood, all cytokines were significantly more elevated in the liver, spleen, and lung 24 h after lipoplex injection (compare FIG. 4 to FIGS. 5-7). As seen in the blood, cytokines decreased 72 h after each injection, and PEGylated lipoplexes generally elicited greater levels of cytokines than that seen with lactose-coated particles. In contrast to blood, all liver, spleen, and lung cytokines were elevated to a greater extent after the second injection. In sharp contrast to the striking increases observed in the liver, spleen, and lung, cytokine levels remained relatively low in the tumor and exhibited concentrations after the second injection that were comparable to that seen after the initial injection (FIGS. 5D, 6D, 7D). The overall lower tumor cytokine levels and notable absence of a further elevation after the second injection is distinctly different from that observed in all other tissues. Consistent with the well-established immunosuppression that is characteristic of the tumor microenvironment, tumor cytokine levels were significantly below that measured in organs and blood in most cases, regardless of coating (Table 1).

One striking observation is that the levels of blood cytokines do not accurately reflect those observed in tissues. In fact, we observed significant differences between blood and tissue cytokines in all organs at all timepoints (see Table 2 for statistical analysis). Furthermore, this difference was noted for all cytokines suggesting that the measurement of blood cytokines that is typically conducted do not accurately reflect innate immune responses at the tissue level (compare FIG. 4 to FIGS. 5-7).

TABLE 1
P VALUES FOR COMPARING TUMOR TO ORGAN CYTOKINE LEVELS

| | Lactose | | | | PEG | | | |
|---|---|---|---|---|---|---|---|---|
| | 1^{st} Injection | | 2^{nd} Injection | | 1^{st} Injection | | 2^{nd} Injection | |
| | Tumor vs. | | | | | | | |
| | 24 | 72 | 96 | 144 | 24 | 72 | 96 | 144 |
| IFN-γ | | | | | | | | |
| Liver | 0.1402 | 0.0450 | 0.0734 | 0.0237 | 0.0336 | 0.0505 | 0.0011 | 0.1311 |
| Spleen | 0.0511 | 0.0007 | 0.0020 | 0.0030 | 0.0100 | 0.0508 | 0.0004 | <0.0001 |
| Lung | 00108 | 0.0226 | <0.0001 | 0.0063 | 0.0715 | 0.0113 | <0.0001 | 0.0068 |
| Blood | 0.0612 | 0.0489 | 0.0820 | 0.4008 | 0.0336 | 0.0505 | 0.0273 | 0.0830 |
| IL-6 | | | | | | | | |
| Liver | 0.0217 | 0.0661 | 0.0108 | 0.0099 | 0.0126 | 0.0153 | 0.0004 | 0.0412 |
| Spleen | 0.0822 | 0.0064 | <0.0001 | 0.0211 | 0.0473 | 0.0073 | 0.0003 | 0.0019 |
| Lung | 0.0805 | 0.0301 | <0.0001 | 0.0001 | <0.0001 | 0.1216 | <0.0001 | 0.0004 |
| Blood | 0.7189 | 0.0161 | 0.0144 | 0.0637 | 0.0311 | 0.7247 | 0.0008 | 0.4888 |
| TNF-α | | | | | | | | |
| Liver | 0.0049 | 0.0512 | 0.0010 | 0.0328 | 0.0098 | 0.0477 | 0.0004 | <0.0001 |
| Spleen | 0.0532 | 0.0063 | 0.0011 | 0.0428 | 0.0268 | 0.0168 | <0.0001 | 0.0007 |
| Lung | 0.0724 | 0.0313 | <0.0001 | 0.0053 | 0.0565 | 0.0304 | 0.0050 | 0.0012 |
| Blood | 0.6052 | 0.0156 | 0.0523 | 0.0175 | 0.0008 | 0.0082 | <0.0001 | 0.0055 |

Underlined values are statistically significant, p < 0.05.

TABLE 2
P VALUES FOR COMPARING BLOOD TO TISSUE CYTOKINE LEVELS

| | Lactose | | | | PEG | | | |
|---|---|---|---|---|---|---|---|---|
| | 1^{st} Injection | | 2^{nd} Injection | | 1^{st} Injection | | 2^{nd} Injection | |
| | Blood vs. | | | | | | | |
| | 24 | 72 | 96 | 144 | 24 | 72 | 96 | 144 |
| IFN-γ | | | | | | | | |
| Liver | 0.0822 | 0.0424 | 0.0657 | 0.0241 | 0.0060 | 0.0106 | <0.0001 | <0.0001 |
| Spleen | 0.0058 | 0.0007 | 0.0020 | 0.0063 | 0.0284 | 0.0262 | 0.0001 | 0.0315 |
| Lung | 0.0073 | 0.0222 | <0.0001 | 0.0035 | 0.0448 | 0.0111 | <0.0001 | 0.0068 |
| Tumor | 0.0612 | 0.0489 | 0.0820 | 0.4008 | 0.0336 | 0.0505 | 0.0273 | 0.0830 |
| IL-6 | | | | | | | | |
| Liver | 0.0300 | 0.0834 | 0.0108 | 0.0011 | 0.0138 | 0.0156 | 0.0004 | 0.0424 |
| Spleen | 0.0865 | 0.0064 | <0.0001 | 0.0213 | 0.0506 | 0.0073 | 0.0003 | 0.0021 |
| Lung | 0.0854 | 0.0308 | <0.0001 | 0.0005 | 0.0005 | 0.1218 | <0.0001 | 0.0018 |
| Tumor | 0.7189 | 0.0161 | 0.0144 | 0.0637 | 0.0311 | 0.7247 | 0.0008 | 0.4888 |
| TNF-α | | | | | | | | |
| Liver | 0.0070 | 0.0586 | 0.0010 | 0.0418 | 0.0142 | 0.0649 | 0.0005 | <0.0001 |
| Spleen | 0.0530 | 0.0066 | 0.0011 | 0.0503 | 0.0308 | 0.0179 | <0.0001 | 0.0006 |
| Lung | 0.0720 | 0.0338 | <0.0001 | 0.0054 | 0.0818 | 0.0343 | 0.0120 | 0.0007 |
| Tumor | 0.6052 | 0.0156 | 0.0523 | 0.0175 | 0.0008 | 0.0082 | <0.0001 | 0.0055 |

Underlined values are statistically significant, p < 0.05.

### Example 4

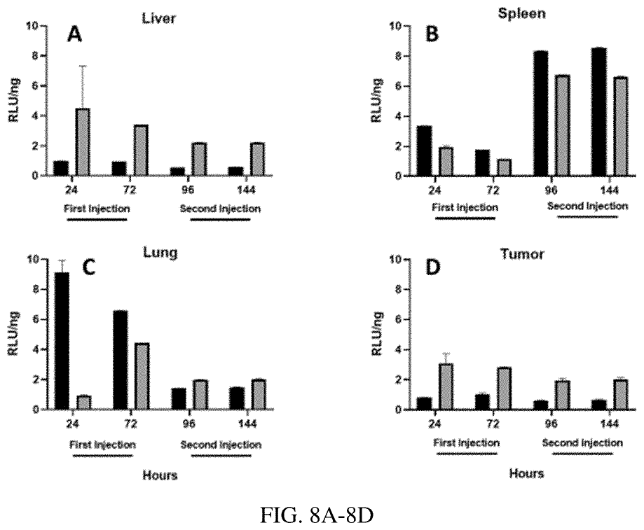

As mentioned above, cytokines (especially interferons) are secreted as part of an antiviral response that functions to combat infection by suppressing viral gene expression. In this context, it is important to note that the plasmid used in our studies contains a mammalian promoter and has been altered to minimize the CpG sequences and reduce immunogenicity. In order to more clearly determine whether reporter gene expression of our plasmid is being affected by changes in tissue cytokines, we utilized our qPCR measurements to calculate the efficiency of expression in individual tissues, i.e., Relative Light Units (RLU)/ng plasmid. As shown in FIG. 8, we observe differences in expression efficiency among tissues, coatings, and injections. More specifically, expression efficiency increased sharply after the second injection in the spleen but decreased in the lung. Surprisingly, the distinct increase in expression efficiency observed in the spleen after the second injection was maintained despite large increases in all three cytokines at 96 h and lower levels by 144 h (compare FIGS. 5-7+8B). While expression efficiency was relatively constant in the liver and tumor, PEGylated lipoplexes consistently resulted in more efficient expression in those organs whereas lactosylated formulations exhibited higher efficiency in the spleen (IG. 8). In contrast to what might be expected if cytokines were involved in suppressing reporter gene expression, expression efficiency in the spleen was highest after the second injection and remained high while all cytokines peaked 24 h after the second injection and subsequently decreased. Taken together, our data are not consistent with a role for tissue cytokines in directly modulating reporter gene expression.

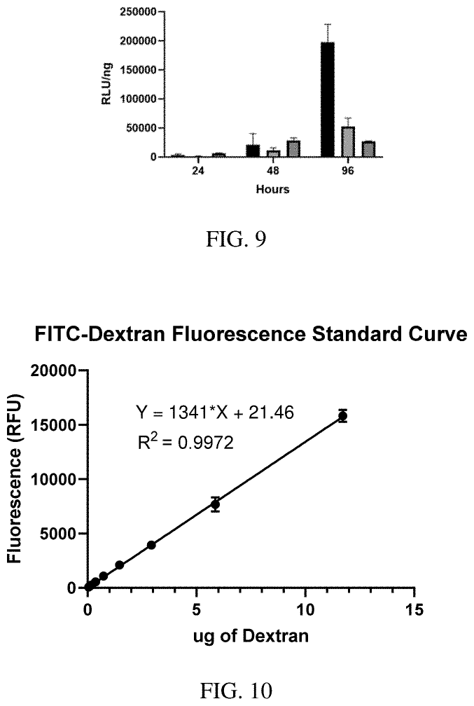

As seen in many gene delivery studies, we observed a poor correlation of plasmid levels with expression in vivo (FIGS. 2+3). This poor correlation was even observed within individual tissues (FIG. 8). In an attempt to better understand these results, we performed in vitro transfection experiments in the same tumor cell line (CT26) that was implanted in our mouse experiments. To be consistent with our in vivo experiments, RTPCR was used to quantify plasmid levels in our cell preparations in which luciferase expression was also quantified at different times after a single treatment with lipoplexes (FIG. 9). It is interesting to note that <10% of the plasmid applied to the cultured cells was present in the cells (after 4 h), and the amount of plasmid (per mg cellular protein) progressively decreased over 96 h (data not shown). In addition, our data are consistent with the well-established abilities of PEG to inhibit interactions with cells and reduce transfection rates. When considered as expression efficiency (i.e., RLU/ng plasmid), it is evident that in vitro expression efficiency increases after 24 h in contrast to the relatively stable tumor values observed in vivo. Another striking difference is that the in vitro values are >1000-fold higher than those observed for the tumor in vivo (compare y axes in FIGS. 8+9). These stark disparities between in vitro and in vivo expression efficiency suggest that the plasmid delivered to a tumor in a mouse exhibits fundamentally different behavior from that within cancer cells in culture.

### Example 5: Effect of Tightening on the Fate of Ferumoxytol, an Imaging Agent

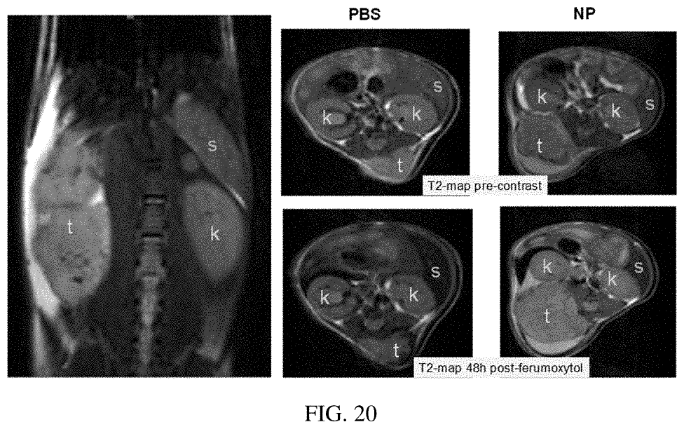

We also assessed the effect of tightening on the fate of ferumoxytol—an FDA-approved iron oxide nanoparticle preparation for intravenous treatment of anemia. Ferumoxytol is also intensively used as an off-label T2-MRI-contrast for vascular imaging as well as “inflammation” imaging, due to its uptake in tumor-associated macrophages and a strong effect on T2/T2* relaxation times Immunocompetent tumor-bearing mice were first pre-treated with either PBS (n=4) or lipoplexes to induce tightening (NP; n=4). T2-weighted abdominal MRI scans were then acquired on a Bruker 9.4 Tesla scanner (FIG. 20, left) in a coronal plane to visualize tumor, spleen and kidney, followed by quantitative T2-MRI maps in the axial plane (FIG. 20, right) for precise calculation of T2 times Immediately after MRI, the animals were infused with 30 mg/kg ferumoxytol and MRI was repeated at 48 hours. The uptake of ferumoxytol in spleen and kidney was significantly diminished after tightening as seen by decreased T2 relaxation times in post-contrast MRI: ΔT2 (T2post−T2pre): −15±2 vs −26±2 ms in the spleen (p=0.005) and −1±3 vs −10±2 ms in the kidney (p=0.02). There were no differences in ΔT2 for tumors in lipoplex vs. vehicle (p=0.4).

### Discussion

It is well established that PEGylated nanoparticles can elicit an immune response that can cause repeat injections to exhibit accelerated blood clearance (ABC). In addition to the potential for ABC to compromise delivery, high levels of pre-existing anti-PEG antibodies have been correlated with life-threatening infusion reactions. However, studies have established that ABC also occurs in immunocompromised animals that are incapable of producing antibodies, demonstrating that the innate immune response is involved in accelerated clearance. Previous work has documented the extensive uptake of naked lipoplexes by circulating immune cells, and our recent work has demonstrated that a lactose coating provides an equivalent reduction in leukocyte uptake and superior tumor accumulation to that observed with PEGylated lipoplexes. We have previously shown that lactosylated lipoplexes elicit less of a cytokine response as compared to PEG, but the results in FIG. 1 clearly indicate that ABC is also observed when lipoplexes are coated with lactose. Because both coatings elicited an elevated systemic cytokine response (FIG. 4), these results are consistent with previous studies showing a role for innate immunity in the ABC phenomenon.

Considering the accelerated clearance discussed above, it would be expected that organ accumulation after the second injection would be limited to the relatively rapid deposition that occurs prior to clearance. Not only did we observe reduced accumulation relative to the initial dose, our data indicate no additional deposition after the second injection in any of the clearance organs suggesting that repeat administration triggers very rapid processing and/or an alternative mechanism that bypasses these organs (FIG. 2). The stimulation of rapid processing by the organs is consistent with the sharp reduction in plasmid accumulation observed in the spleen and lung after repeat administration, but a similar reduction was not evident in the liver. This difference among these organs could potentially be explained by previous studies showing that prolonged presence in the liver indicates delivery to hepatocytes as opposed to macrophages. It is important to note that we have previously observed considerable accumulation in each of these organs with uncoated lipoplexes that are cleared much more rapidly than the coated formulations used in this study. Therefore, we conclude that the circulation time is sufficient to allow organ accumulation even under accelerated clearance conditions, yet we do not observe additional plasmid in liver, lung, or spleen after the second dose. In contrast to that observed in the clearance organs, additional accumulation is definitely evident in the tumor after repeat injection, further demonstrating that circulation times after the second injection are sufficient to allow supplemental deposition (FIG. 2). Accordingly, it is unclear if particle deposition after the second injection is preferentially cleared and/or if lipoplex uptake by these organs is inhibited during repeat administration.

Changes in reporter gene expression correlated poorly with changes in plasmid accumulation in the clearance organs (liver, spleen, and lung), but a better correlation was observed in the tumor (compare FIGS. 2+3). Considering previous work showing that cytokines can suppress gene expression, we performed ELISA assays to monitor the three main cytokines that have been correlated to changes in reporter gene expression. Although levels of specific cytokines can vary in response to foreign material, the levels of IFNγ, IL-6, and TNFα exhibited very similar trends and thereby serve as general indicators of the cytokine response. Unlike most prior studies, we measured both blood and tissue cytokines, and we observed striking differences, i.e., blood levels were generally 10-1000-fold lower than that seen in tissues and exhibited different trends (compare FIGS. 4-7; see Table 2 for statistical analysis). These sharp differences between blood and tissue cytokines suggest that nanoparticle administration elicits disparate responses at the systemic and tissue level. However, the levels of cytokines in the tissue did not correlate with expression even after accounting for changing plasmid levels (FIG. 8), suggesting that other mechanisms are more directly involved in controlling reporter gene expression. This is best illustrated by the fact that expression efficiency in the tumor was comparable to or below that in other organs despite tumor cytokines being maintained at much lower levels throughout our study. Because cytokine production by infected cells is thought to be indicative of an antiviral response, our data may indicate that the plasmid alterations used in our study (mammalian promoter, minimal CpG) may be sufficient to circumvent these silencing mechanisms.

In conclusion, our results showing a distinct cytokine response to intravenous lipoplex administration consistent with previous studies suggesting a role for an innate immune response in the accelerated blood clearance phenomenon observed after repeat administration. In addition to reduced circulation times, this study documents a dramatic difference between the first and second intravenous injection in terms of biodistribution, expression, and cytokine response, and this was observed with lipoplexes coated with either lactose or PEG. The inability of the second injection to increase plasmid levels in the liver, lung, and spleen differs from our previous studies using uncoated lipoplexes, indicating that a response to the hydrophilic coating may contribute to these effects. The fact that additional accumulation was evident in the tumor after repeat injection suggests that the immune response to our lipoplexes includes mechanisms that reduce uptake in clearance organs. This may be an important consideration when developing nucleic acid-based therapeutics for treating these organs (e.g., liver diseases), and prolonged dosing intervals may allow this response to dissipate. In this context, it is noteworthy that Onpattro® (an approved siRNA product targeting the liver) is dosed at 3-week intervals. Conversely, the persistent ability of the tumor to accumulate lipoplexes may selectively permit higher expression to be achieved by repeat injection (relative to organs), consistent with our previous findings. While the use of a lactose coating generally reduces the cytokine response as compared to PEGylated lipoplexes, changes in expression did not correlate with either blood or tissue cytokine levels. Surprisingly, the levels of blood cytokines do not correlate with tissue cytokines and are generally 100-fold lower. This suggests that the systemic immune response can differ markedly from that in individual tissues, which may be an important consideration for tissue-specific therapies. It is worth noting that we observe relatively constant expression efficiency in all tissues in contrast to previous studies that reported up to 100-fold greater efficiency in the tumor.

Taken together, our results demonstrate that repeat injection of lipoplexes elicits an immune response that dramatically alters delivery, consistent with the well-established refractory period described in other studies. These prior studies attributed the inability of repeat dosing to enhance luciferase activity to the production of inflammatory cytokines, and our results indicate that mechanisms that directly restrict particle uptake contribute to the refractory period.

### Materials and Methods for Examples 5 Through 12

### Liposome/Lipoplex Preparation

Sphingosine, cholesterol, 1,2-diarachidoyl-sn-glycero-3-phosphocholine (DAPC), and C16-PEG750-Ceramide were purchased from Avanti® Polar Lipids (Alabaster, Ala.) and used to prepare liposomes at a 3:2:4.5:0.5 mole ratio (respectively) as previously described. The liposomes preparations have an average diameter of 135 nm, a PDI of 0.137, and were prepared in 5% dextrose to adjust the tonicity of the injection solution. To form lipoplexes, liposomes were mixed with plasmid DNA (donated from Megabios Corp., Burlingame, Calif.) in 5% dextrose at a charge ratio of 0.5. The resulting lipoplexes have a diameter of 176 nm, a PDI of 0.084. A Malvern Zetasizer Nano-ZS was used for all size measurements. Phosphate buffered saline (PBS; 10 mM phosphate, 137 mM NaCl, 2.7 mM KCl, pH 8.25), lipoplexes [0.075 pmoles of DNA (25 μgrams) complexed with 0.125 pmoles total lipid (0.0375 μmoles sphingosine)], or naked liposomes (0.125 μmoles total lipid) were injected via tail vein as previously described.

### Animals

Tumor Bearing Experiments: Prior to treatment, female immunocompetent Balb/c mice 6-10 weeks old were acquired from Jackson labs (Bar Harbor, Me.) and allowed to acclimate for one week. The mice were then inoculated in the right flank with approximately 1 million CT26 murine colon carcinoma cells (ATCC #CRL-2638). Tumors were allowed to grow for approximately 7 days or until tumor size reached at least 100 mm³. Each mouse received a single dose of 200 μL 1×PBS, liposomes, lipoplexes (25 pg total DNA), or 1 pg of recombinant mouse IFN-λ2 (R&D Systems, 4635-ML-025/CF) in 100 μL 1×PBS by a tail vein injection. Twenty-four hours after pre-treatment, animals were administered a 10 mg/kg dose of FITC-dextran (Sigma-Aldrich, FD150S) or 10 mg/kg dose of liposomal doxorubicin (Doxil®) via tail vein. Twenty-four hours after the dextran/Doxil® injections, animals were euthanized, and organs collected for dextran/drug accumulation analysis.

All animal procedures were approved by the University of Colorado Institute for Animal Care and Use Committee in accordance with guidelines from the National Institutes of Health (USA).

### Quantification of FITC-Dextran Fluorescence and Dextran Extraction Efficiency

FITC-dextran was chosen for these studies since it is commonly used to quantify endothelial and epithelial cell barrier permeability. The physical properties of dextran have been extensively characterized, and higher molecular weight dextrans possess progressively larger hydrodynamic diameters. FITC-dextran with a molecular weight of approximately 150 kD was used for these studies, and dynamic light scattering measurements estimate its hydrodynamic diameter at approximately 100 nm (data not shown); consistent with published data approximating the size of various molecular weight dextrans.

A standard curve of fluorescence for the FITC-dextran used in this project was determined through fluorescence measurements of a serially diluted stock solution of the FITC-dextran in 1×PBS. Fluorescence measurements were conducted in a CellStar™ μClear™ 96-Well, Cell Culture-Treated, Flat-Bottom Microplate from Greiner Bio-One™ (Monroe, N.C.) at an excitation of 490 nm and emission of 520 nm using a Molecular Devices (San Jose, Calif.) SpectraMax® MS. To determine dextran extraction efficiency for each whole organ and tissue (lung, heart, liver, spleen, kidneys, brain, tumor), varying amounts of dextran (i.e., 0, 1, 2, 3, 4, 5 μg) were spiked into sets of blank tissues from untreated mice. The tissues were then processed through the dextran extraction procedure described below. Fluorescence of the extracted material was then measured, and total micrograms of dextran calculated with the fluorescence standard curve. Each extraction from spiked tissues was performed in duplicate. The resulting values were averaged and plotted against the total micrograms of dextran spiked into the tissues to create a standard curve for dextran extraction.

### Extraction of FITC-Dextran and Quantification of Organ Accumulation

Organs collected from animals were immediately flash-frozen in liquid nitrogen and stored at −80° C. until analysis. Organs were thawed, weighed, and extracted using a PBS buffer extraction method. Briefly, each organ was placed in a 2-ml screw cap tube (each liver was equally divided into two tubes) with five 2.3-mm diameter zirconia/silica beads from BioSpec Products (Bartlesville, Okla.) plus 1 mL of lysis buffer (1×PBS with 1% sodium dodecyl sulfate), and 200 μg Proteinase K from Qiagen (Hilden, Germany) was added to each organ. Organs were then incubated at 55° C. for 1 hour. After incubation, organs were homogenized using a MiniBeadBeater-16 Model 607 from BioSpec Products (Bartlesville, Okla.) for 3.5 minutes. Once homogenized, 100 μL of 20% w/v trichloroacetic acid was added to the homogenate and samples were vortexed for 10 seconds to precipitate proteins. The homogenate was then centrifuged at 15,000 RCF for 10 min. The supernatant was aspirated into a new tube (supernatants of the divided livers were combined and total volume brought up to 10 mL with 1×PBS) and solution pH was adjusted to pH 8-9 using 1 M sodium hydroxide. FITC fluorescence of the extracted solution was then measured, and total dextran accumulation was calculated using the extraction efficiency and standard curves described above.

### Measurement of Plasmid Blood Levels

To monitor plasmid levels in the blood, mice were bled (15-50 μl) at 60 minutes using their submandibular veins as previously described. Whole blood was collected and centrifuged (2,000×g for 10 minutes) to separate plasma from the blood cell fraction, and each sample was prepared with a Qiagen DNeasy® Blood and Tissue kit (Qiagen, Germantown, Md.). Both the plasma and cell pellet were treated with the included lysis buffer per manufacturer's instructions. The samples were then subject to quantitative PCR using a QuantiTech® RTPCR Kit (Qiagen, Germantown, Md.) on an Applied Biosystems 7500 RTPCR instrument (Grand Island, N.Y.). A standard curve of pure plasmid was used to determine quantity. Although our PCR measurements do not directly determine whether the plasmid is associated with the lipoplex, previous studies have shown that free plasmid is completely degraded (i.e., not detectable) within 10 min of intravenous administration in a mouse, and thus we assume that the reported levels of plasmid predominantly represent plasmid associated with lipoplexes.

### Determination of Plasmid Levels in Tissues

To determine delivery of plasmid DNA to mouse tissues, animals were sacrificed 24 or 72 h after each lipoplex administration, and extracted organs were harvested and flash frozen in liquid nitrogen. Organs were subsequently thawed, weighed, and DNA was extracted using the Qiagen DNeasy® Blood and Tissue Kit (Qiagen, Germantown, Md.) as described above. Quantitative PCR (qPCR) was performed on tissue samples using QuantiTech® RTPCR Kit (Qiagen, Germantown, Md.) on an Applied Biosystems 7500 RTPCR instrument (Grand Island, N.Y.). A standard curve of pure plasmid was used for quantification in addition to amplicon efficiency factors that account for different efficiencies of amplification. We have previously determined the extraction efficiencies of plasmid from isolated organs, and these values were used to calculate the plasmid levels depicted in the results. It should be noted that the amplified PCR fragment is 1072 base pairs representing approximately one-sixth of the plasmid sequence. Therefore, our methods cannot distinguish between intact plasmid and large fragments that encode both primer sites. Previous studies have shown that free plasmid is rapidly degraded in tissues, and thus the presence of large fragments would be unlikely at the time points (24 and 72 h) used in our experiments.

### Cytokine Response

A separate set of tumor-bearing mice was used to quantify cytokine levels after repetitive injection of lipoplexes. In addition to the coated lipoplex formulations described above, mice were treated with phosphate buffered saline as a negative control. In these studies, triplicate mice were treated with lipoplex formulations as described above, and blood was collected 24 and 72 h after both the first and second injection of lipoplexes (3-day interval). Serum samples were allowed to clot for 30 minutes and spun at 2,000×g for 15 minutes per manufacturer's instructions (R&D Systems™, Minneapolis Minn.). For solid tissues (liver, lung, spleen, tumor), extracted tissues were homogenized in buffer and cytokines extracted/quantified as described by the manufacturer. Samples were assayed in triplicate for interferon gamma (IFN γ), interleukin 6 (IL-6), and tumor necrosis factor alpha (TNFα) and using ELISA kits purchased from R&D Systems™ (Minneapolis Minn.). The limits of detection of IFNγ, IL-6, and TNFα are 2, 1, and 7 pg/mL for blood and 20, 40, 140 pg/g for tissues, respectively.

### Quantification of Doxorubicin Fluorescence and Organ Accumulation

A standard curve of fluorescence for doxorubicin was determined through fluorescence measurements of a serially diluted stock solution of the Doxil® in 90% isopropyl alcohol (IPA) acidified with HCl to a final concentration of 0.075 M. Fluorescence measurements were conducted in a CellStar™ μClear™ 96-Well, Cell Culture-Treated, Flat-Bottom Microplate from Greiner Bio-One™ (Monroe, N.C.) at an excitation of 470 nm and emission of 595 nm using a Molecular Devices (San Jose, Calif.) SpectraMax® M5. To determine doxorubicin levels in whole organs and tissues (lung, heart, liver, spleen, kidneys, brain, tumor, skin, plasma), blank tissues were run through an extraction procedure using 90% IPA/0.075 M HCl as described below. Once extracted, the blank organ extract was used as a buffer to serially dilute a stock solution of Doxil®. The equation from the resulting standard curve of fluorescence from each specific organ was used to calculate the total amount of doxorubicin extracted from a corresponding Doxil® treated organ.

Extraction of Doxorubicin from Tissues

Organs collected from animals were immediately flash-frozen in liquid nitrogen and stored at −80° C. until analysis. Organs were thawed, weighed, and extracted using a buffer made of 90% IPA/0.075 M HCl. Briefly, each organ was placed in a 2-ml tube containing lysis matrix A (MP Biomedical™, 6910500) (each liver was equally divided into four tubes) and 1 mL of 90% IPA/0.075 M HCl was then added to each tube. Organs were then homogenized using a MiniBeadBeater-16 Model 607 from BioSpec Products (Bartlesville, Okla.). Tubes were shaken for 90 second then allowed to rest on ice for 5 minutes before another 90 second shake. Once homogenized, the tubes were allowed to rest on ice for 5 minutes before being centrifuged at 15,000 RCF for 15 minutes at 4° C. The supernatant was aspirated into a new tube and total volumes for all organs, except livers, were standardized to 1 mL using 90% IPA/0.075 M HCl. Divided liver supernatants were added together and standardized to 5 mL using 90% IPA/0.075 M HCl. Doxorubicin fluorescence of the extracted solution was then measured, and total doxorubicin accumulation was then calculated using the doxorubicin fluorescence standard curves described above. An extraction efficiency of 95% was assumed for all extractions (Gabizon, A. A., 1992, Cancer Res 52, 891-896).

### Example 6: Extraction Efficiency of FITC-Dextran in Tissues

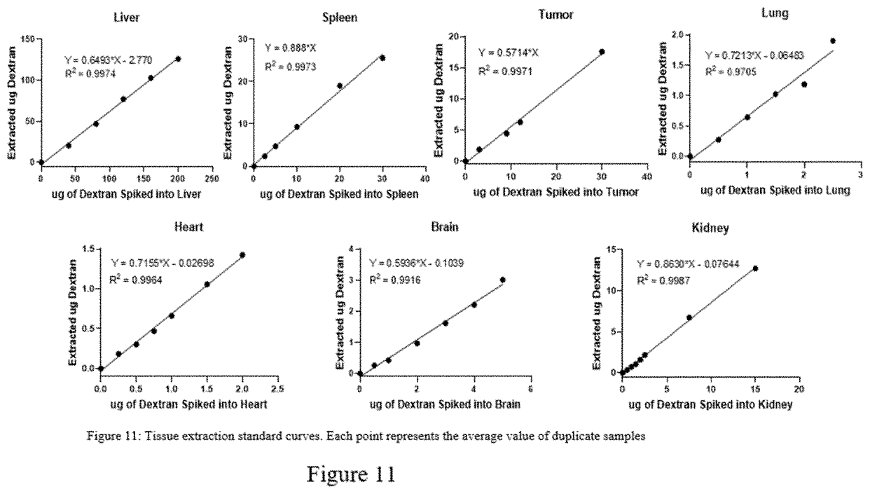

FITC-Dextran was added to 1×PBS and serially diluted to obtain a standard curve of FITC fluorescence in PBS. As shown in FIG. 10, a direct correlation was observed between the amount of dextran in solution and FITC fluorescence. The lowest level of detection from the standard curve was 0.0048 ng of FITC-dextran per microliter of 1×PBS (pH 8.25). Tissues from untreated mice were injected with known amounts of FITC-dextran, and fluorescence of the extracted material was measured as described above. To assess extraction efficiency, the fluorescence of extracted material from each spiked tissue was converted to μg of dextran using the standard curve of FITC fluorescence. The spiked amount of dextran was then plotted against the extracted amount of dextran to obtain a standard curve (FIG. 11). The average extraction efficiencies from the different tissues varied from 51-92% as shown in Table 3 and were used to calculate total dextran accumulation in each tissue.

TABLE 3

| Tissue | Average Extraction Efficiency %: |
|---|---|
| Liver (n = 12) | 60.09 ± 0.06 |
| Spleen (n = 12) | 92.85 ± 4.68 |
| Lung (n = 12) | 64.39 ± 9.19 |
| Kidney (n = 16) | 78.21 ± 11.36 |
| Heart (n = 14) | 67.54 ± 5.23 |
| Brain (n = 14) | 51.08 ± 7.72 |
| Tumor (n = 10) | 55.49 ± 5.80 |

### Example 7: Effect of Lipoplex Pretreatment on Dextran Accumulation and Tumor-to-Organ Accumulation Ratio

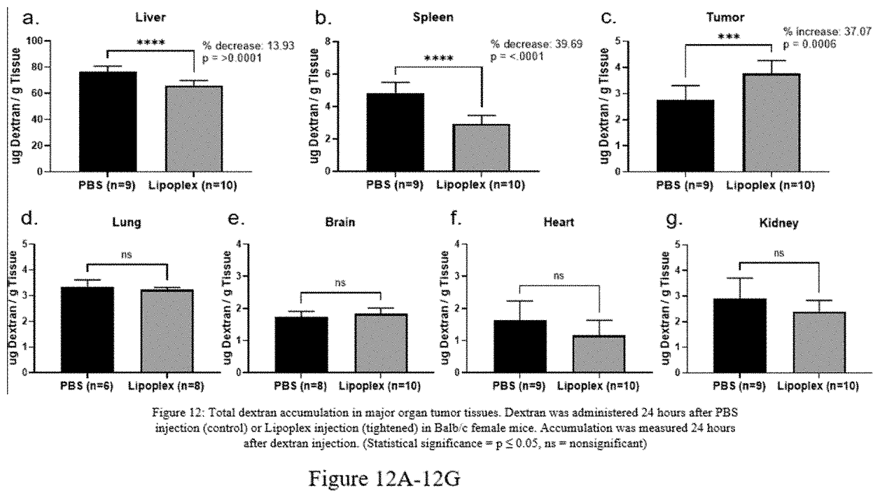

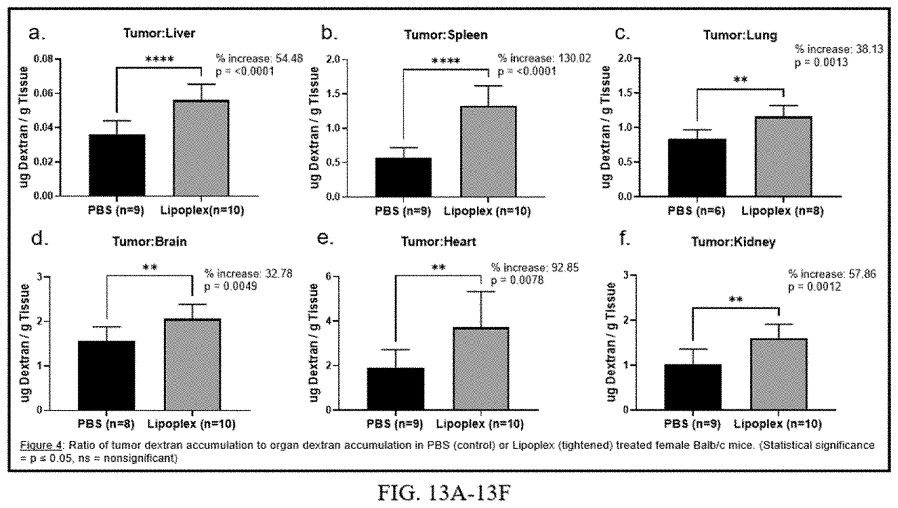

To assess the effects of a lipoplex pretreatment on dextran accumulation, mice were intravenously injected with either PBS or lipoplexes. Twenty-four hours after pretreatment, a 10 mg/kg dose of FITC-dextran was administered. After an additional 24 hours following dextran administration, tissues were harvested from experimental mice and the total dextran accumulation in tissues was determined using the standard curves shown in FIGS. 10+11. Our results demonstrate that a lipoplex pretreatment significantly reduces dextran accumulation in liver and spleen when compared to mice pretreated with PBS (FIGS. 12A+B). We also observe a reduction in dextran accumulation for other major organs (lung, heart, kidney) although these trends are not statistically significant under our experimental conditions (FIGS. 12 D-G). In contrast to that seen in the major organs, the tumor experienced a significant increase in dextran accumulation when compared to mice pretreated with PBS (FIG. 12C). When these results are computed as a ratio between tumor and organ dextran accumulation, the effect of the lipoplex pretreatment becomes more evident (FIG. 13). More specifically, all major organs from mice pretreated with lipoplexes show a significant increase in the tumor-to-organ accumulation ratio when compared to tissues from mice pretreated with PBS (FIG. 13).

The immune response to lipoplexes results in epithelial tightening which significantly reduces uptake by the major clearance organs (liver, spleen) and consequently increases accumulation in the tumor. The dramatic effects of epithelial tightening are most evident when the results are considered as a ratio of tumor-to-organ accumulation. We observe a 60% and 130% increase in the tumor:liver and tumor:spleen accumulation, respectively. We also observe a significant (>75%) increase when tumor accumulation is considered relative to the lung, heart, and brain, indicating that tightening is a systemic response exhibited by the epithelium associated with major organs but not the tumor.

### Example 8: Dependence of Dextran Accumulation on the Presence of Nucleic Acids in the Pretreatment Injection

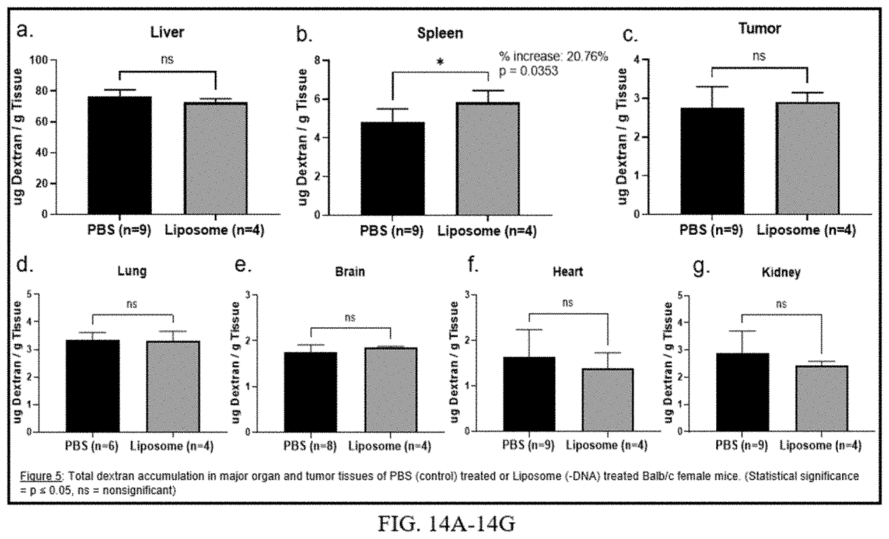

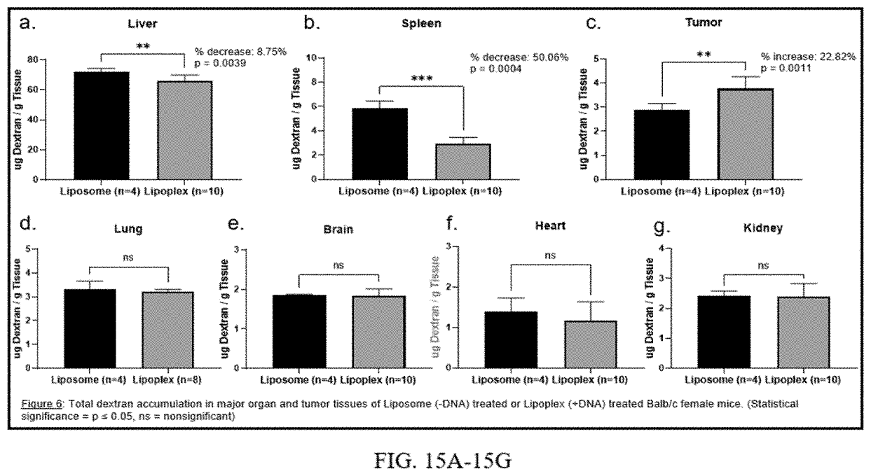

Several recent studies have shown that saturation of the MPS leads to greater circulation times and enhanced delivery of subsequently administered nanoparticles to target tissues. However, this saturation strategy requires overloading the body's primary filtration systems with excessive amounts of “decoy” material. To determine if the MPS saturation contributes to the refractory/tightening response to lipoplex pretreatment that we observe, we compared the effects of liposome (lipid nanoparticle without DNA) versus lipoplex pretreatments. Dextran accumulation in organs and tumor tissues were compared between PBS, liposome, or lipoplex pretreatment groups. The liposome formulation and sizes were identical to the lipid nanoparticles used to form the lipoplexes and were not complexed with plasmid DNA. The timing of the pretreatment and dextran administrations were the same as described above. Our results show that there are no significant differences in dextran accumulation in the organs and tumor between liposome and PBS pretreatments except in the spleen which exhibited a slight increase for the liposome-treated group (FIG. 14). In contrast, lipoplex pretreatment significantly reduced dextran accumulation in the liver and spleen compared to animals pretreated with liposomes (FIGS. 15A+B). We also observe slightly lower accumulation in the lung, brain, heart, and kidneys from lipoplex-treated animals as compared to those receiving liposome pretreatment, but the differences did not achieve statistical significance (FIGS. 15D-G). However, dextran accumulation in tumors was significantly increased in lipoplex-treated mice compared to liposome-treated mice.

### Example 9: Tightening Response is Maintained for at Least 72 Hours

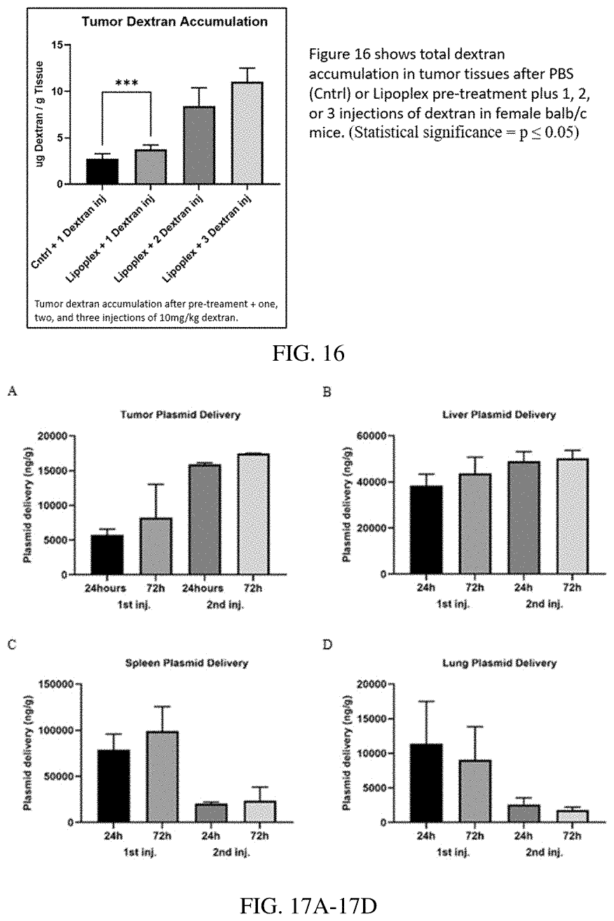

Because chemotherapy involves chronic dosing, it will also be important to determine the duration of the tightening response and how this might be exploited during repeat dosing. The data in FIG. 16 indicate that the improved tumor accumulation we observe after a lipoplex injection is maintained over at least three dextran doses given at 24 h intervals. More specifically, no significant difference in tumor-to-liver accumulation ratio was observed among the three injections. These results demonstrate how epithelial tightening can be more fully exploited during repetitive dosing to achieve high tumor accumulations. Furthermore, these findings suggest that tightening elicited in response to lipoplexes is maintained for at least 72 hours.

### Example 10: Repeat Lipoplex Administrations Result in Enhanced Accumulation of Nucleic Acid in the Tumor after a Second Injection without Significant Accumulation in Major Organs

Early gene therapy studies using intravenously-administered non-viral vectors made the observation that expression was not enhanced upon repeat administration. The researchers reported a “refractory period” of several weeks during which repeat injections could not enhance gene expression. This effect has also been observed in human clinical trials. Furthermore, the duration of the refractory period was correlated with the dose of DNA and was shortened by decreasing the dose of plasmid. These effects on transgene expression were mainly attributed to immunostimulatory effects of CpG sequences, but this effect was not abolished in studies where CpG plasmids were used. In addition, similar effects were observed in studies using polymer-based non-viral vectors administered via intranasal and pulmonary delivery, suggesting that this effect involves a universal mechanism that is independent of the route of administration. Nonetheless, previous studies all assumed that the limitations of repeat dosing of non-viral vectors arose from intracellular mechanisms that inhibited transgene expression; it was inconceivable that uptake could be directly affected by a dose given days prior. Unlike prior work, our studies of repeat administration included a quantification of plasmid delivered to each organ as well as expression. In our study, four doses of lipoplexes were administered at 3-day intervals, and we observed minimal increases of plasmid accumulation in the major organs but significant enhancement (26-fold!) in delivery to the tumor.

Results presented here have quantified the effects of repeat lipoplex administration. In these experiments, tumor-bearing mice were intravenously administered a dose of lipoplexes, and plasmid accumulation was monitored by quantitative PCR after 24 and 72 hours. After 72 hours, a second dose of lipoplexes was administered and plasmid accumulation was again monitored 24 and 72 hours after the second dose. As shown in the top left panel of FIG. 17, the second injection resulted in additional plasmid accumulation in the tumor as might be expected. In contrast, additional plasmid accumulation was not observed in the liver, spleen or lungs, consistent with the refractory period described in previous work. Furthermore, the increase in tumor accumulation was greater than two-fold, suggesting that the decreased uptake by the organs enhances delivery to the tumor (FIG. 17).

These data are in agreement with our previous studies on repeat lipoplex administration, and consistent with our dextran experiments demonstrating that the tightening response selectively reduces paracellular permeability in organs but enhances accumulation in the tumor. In addition, this effect could not have been observed in the decades of studies utilizing liposomal drug delivery systems because epithelial tightening is an anti-viral response that is not elicited in response to naked liposomes.

### Example 11: Tumor in Immunosuppressed State

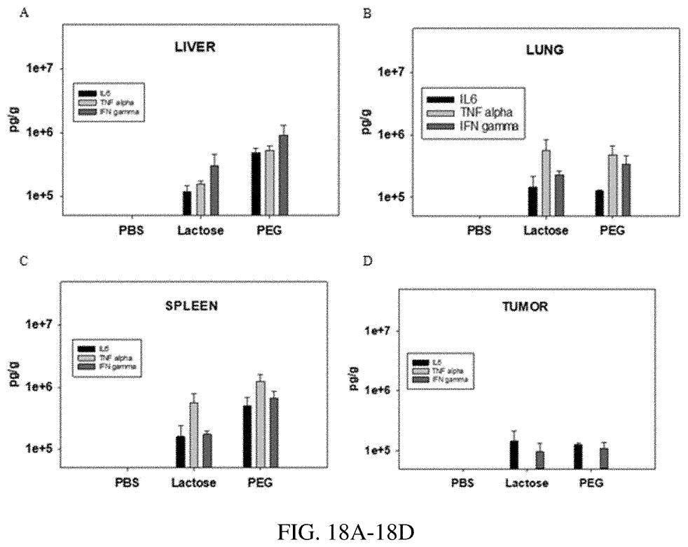

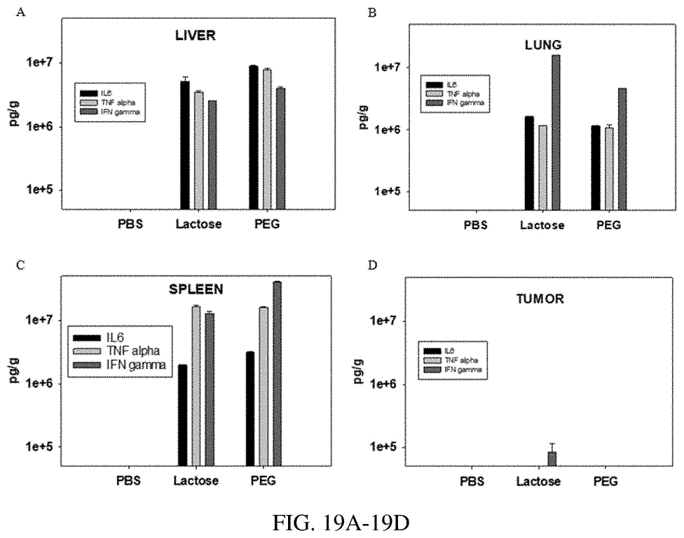

Although the ability to restrict epithelial permeability makes intuitive sense in terms of evolving a mechanism to reduce viral infection, it is surprising that this response is not exhibited in the tumor. We reasoned that because innate immune responses are transmitted via cytokines, it was feasible that the immune-suppressed state of an established tumor might result in perturbed signaling that prevents the associated epithelium from initiating the tightening response. To assess this possibility, separate sets of mice were administered repeat doses of lipoplexes as described above for FIG. 17. The animals were sacrificed 24 and 72 hours after each of the two injections, and cytokine levels in the blood, liver, spleen, lungs, and tumor were quantified via ELISA assay. Three cytokines (IFN-γ, IL-6, TNF-α) that are indicative of the innate immune response and have been implicated in responses to repeat lipoplex injection were monitored in immunocompetent Balb/c mice. Separate sets of mice injected with PBS instead of lipoplexes were used to control for any responses to intravenous injection. In general, cytokine levels were highest after the second injection and decreased from 24 to 72 h after each injection (FIGS. 18+19). All three cytokines showed similar patterns, and cytokine levels in the organs were consistently 10-100 fold higher than that observed in the blood. As shown in FIG. 18, levels of all three cytokines were elevated 100-1000 fold in organs from mice injected with lipoplexes as compared to saline. In contrast, cytokine levels in the tumor were barely detectable and only slightly elevated (<10-fold), consistent with the immunosuppressed state of the tumor.

### Example 12: Effect of IFN-λ on Epithelial Tightening

Although our experiments did not specifically measure IFN-λ levels, the consistent cytokine response exhibited by IFN-γ, IL-6, and TNF-α we reasoned that IFN-2. levels would also be minimal in the tumor and that low levels of cytokines (e.g., IFN-λ) might prevent the tightening response in the tumor-associated epithelium, thereby allowing delivery to the tumor despite endothelial tightening in other vasculature. Preliminary studies using direct injection of IFN-γ into tumors demonstrate that cytokine levels are maintained for at least 4 h (data not shown). Not surprisingly, researchers have yet to identify all the molecules involved in triggering/signaling endothelial/epithelial tightening, but a newly-discovered interferon, IFN-λ, appears to play a critical role. In contrast to inflammatory cytokines (e.g., TNF-α, IFN-γ, IFN-α, IFN-β) that increase vascular permeability, IFN-λ works specifically on the endothelial/epithelial cells to strengthen barrier integrity and prevent viral invasion. Although all the molecules involved in signaling endothelial tightening have yet to be identified, we surprisingly discovered that the immunosuppressive environment within the tumor suppresses the secretion of signaling molecules and effectively blocks the tightening response. Ultimately, the lack of a tightening response in the tumor epithelium allows nanoparticles unaltered access to the tumor upon repeat administration.

As described above, our experiments indicate that epithelial tightening can be induced by intravenous lipoplex administration, and this response can significantly curtail nanoparticle accumulation upon subsequent administrations. The fact that tumor accumulation is unaffected or enhanced provides a counter-intuitive approach to improving nanoparticle-mediated cancer therapies. If epithelial tightening can be elicited by an initial lipoplex dose, it should be possible to target subsequent therapeutic administrations to the tumor while minimizing deposition in other tissues. Using this approach, the tightening induced by lipoplexes is then followed by therapeutic doses of anti-cancer agents that preferentially accumulate in the tumor, thereby greatly reducing off-target effects.

### Materials and Methods for Examples 13 and 14

Liposome/Lipoplex Preparation: Sphingosine, cholesterol, 1,2-diarachidoyl-sn-glycero-3-phosphocholine (DAPC), and C16-PEG750-Ceramide were purchased from Avanti® Polar Lipids (Alabaster, Ala.) and used to prepare liposomes at a 3:2:4.5:0.5 mole ratio (respectively) as previously described 1. The liposomes preparations have an average diameter of 135 nm, a PDI of 0.137, and were prepared in 5% dextrose to adjust the tonicity of the injection solution. To form lipoplexes, liposomes were mixed with plasmid DNA (donated from Megabios Corp., Burlingame, Calif.) in 5% dextrose at a charge ratio of 0.51 (Betker, J. L. & Anchordoquy, T. J. Effect of charge ratio on lipoplex-mediated gene delivery and liver toxicity. Ther Deliv 6, 1243-1253 (2015).

The resulting lipoplexes have a diameter of 176 nm, a PDI of 0.084. A Malvern Zetasizer Nano-ZS was used for all size measurements. 5% dextrose, lipoplexes [0.075 pmoles of DNA (25 μgrams) complexed with 0.125 pmoles total lipid (0.0375 pmoles sphingosine)], or naked liposomes (0.125 μmoles total lipid) were injected via tail vein as previously described (Betker, J. L. & Anchordoquy, T. J, 2015. Mol Pharm 12, 264-273).

Animals Prior to treatment, female immunocompetent Balb/c mice 6-10 weeks old were acquired from Jackson Labs (Bar Harbor, Me.) and allowed to acclimate for one week. Each mouse received a single dose of 200 μL 5% dextrose, lipoplexes, or liposomes by a tail vein injection. 12, 24, 36, 48, and 72 hours after injections, animals were euthanized, and whole blood collected through cardiac puncture for serum preparation and ELISA analysis. All animal procedures were approved by the University of Colorado Institute for Animal Care and Use Committee in accordance with guidelines from the National Institutes of Health (USA).

IL28A/B Quantification by ELISA: Serum was prepared by allowing whole blood to clot at room temperature for 30 minutes. Clotted blood was then centrifuged at 5000 RCF for 15 minutes at 4° C. Serum was then aspirated into new tubes and frozen at −20° C. Serum levels of IFN-λ were determined through enzyme-linked immunosorbent assay (ELISA) using the manufacturer's recommendations and protocols. Mouse IL-28A/B (IFN-lambda 2/3) DuoSet® ELISA (DY1789B-05) was purchased from R&D Systems® (Minneapolis, Minn.).

Interferon Lambda Receptor 1 (IFNλR1) mRNA Expression Quantification by Reverse Transcription-Polymerase Chain Reaction (RT-qPCR): Human Lung Microvascular Endothelial Cells (540-05A) (HLMVECs) purchased from Sigma-Aldrich® (Burlington, Mass.) were cultured and left untreated or treated with 100 ng/mL recombinant human IL-29 (R&D Systems® 1598-IL-025) or 100 ng/mL IL-28A/B (R&D Systems® 8417-IL-025/5259-IL-025) for 24 hours. Human Liver Epithelial Cells (H-6044) purchased from Cell Biologics, Inc (Chicago, Ill.) were cultured and left untreated or treated with 100 ng/mL recombinant human IL-29, 100 ng/mL IL-28A/B, or 1 μg/mL of lipoplexes for 24 hours. After 24 hours, using TaqMan™ Fast Advanced Cells-to-CT™ kit purchased from Invitrogen™ (Waltham, Mass.), cells were collected according to manufacturer recommendations and IFNλR1 expression was quantified. PCR assay ID Hs00417120_ml (ThermoFisher Scientific™, Agawam, M A) was used to quantify IFNλR1 gene expression and PCR assay ID Hs03928985_g1 (ThermoFisher Scientific™, Agawam, M A) was used to quantify reference gene RNA18S5 expression.

### Example 13: Effect of Lipoplex Treatment on IFN-λ Response

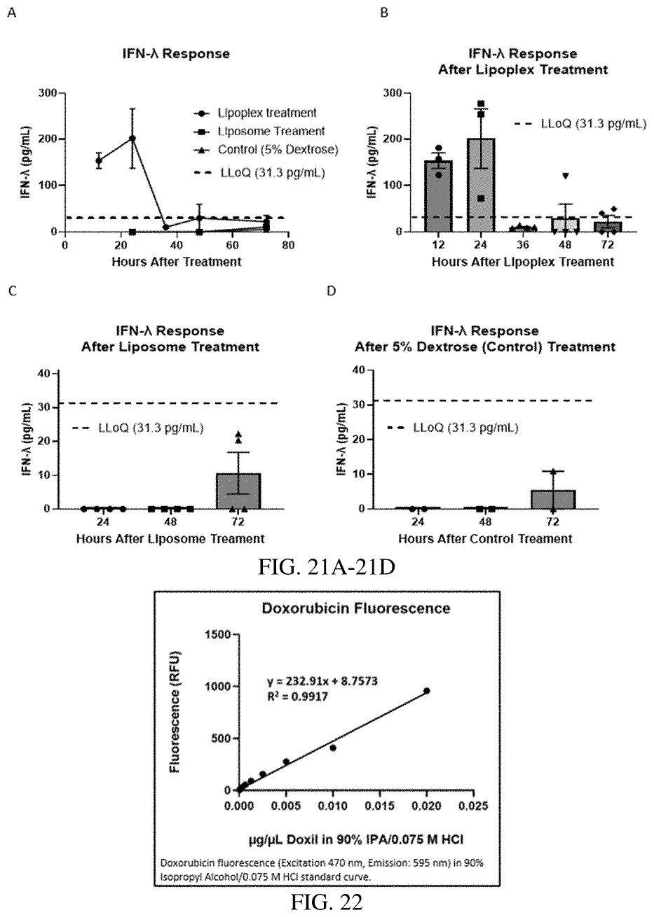

To determine if lipoplex treatment initiates a systemic IFN-λ response, female Balb/c mice were injected with lipoplexes, liposomes, or 5% dextrose as a control. Serum was collected from the mice at several time points up to seventy-two hours. IFN-λ levels in serum were then quantified using enzyme-linked immunosorbent assay (ELISA) (FIG. 21). Serum from mice treated with lipoplexes show an average of 153.67 pg/mL of IFN-λ twelve hours after treatment (FIG. 21B). Twenty-four hours after lipoplex treatment, serum levels of IFN-λ peak at an average of 201.81 pg/mL. At 36 hours after lipoplex treatment, serum levels of IFN-λ were detected at an average of 10.40 pg/mL. However, this is well below the lowest concentration that can be reliably determined by the ELISA (Lower Limit of Quantification (LLoQ)=31.30 pg/mL). Forty-eight hours after lipoplex treatment only 1 out of 4 mice showed detectable levels of IFN-λ at 119.90 pg/mL. At the final time point of 72 hours, two out of four mice showed detectable levels of IFN-λ at 40.27 and 49.57 pg/mL (FIG. 21B). For liposome and 5% dextrose treatments, no serum samples showed IFN-λ at levels above the LLoQ at any time point. This data shows that lipoplex treatment does initiate a systemic IFN-2 response that peaks 24 hours after injection while liposome treatment does not produce any IFN-λ. It can be concluded that the nucleic-acid/lipid complex of lipoplexes is the primary driver of the IFN-λ response to lipoplex treatment.

To test the hypothesis that microvascular endothelial cells and liver epithelial cells respond to IFN-λ, real-time quantitative polymerase chain reaction (RT-qPCR) analysis was used to determine Interferon Lambda Receptor 1 (IFN λR1) gene expression in primary Human Lung Microvascular Endothelial Cells and primary Human Liver Epithelial Cells (TABLES 4 and 5). Surprisingly, no expression of IFN λR1 was detected in basal state endothelial cells (TABLE 5). There was also no expression detected in endothelial cells treated with IFN-λ1 or IFN-λ2/3 (TABLE 5). Human Liver Epithelial Cells show expression of IFN λR1 at basal state and show a fold-change increase in gene expression of 2.132 when treated with IFN-2d, a fold-change increase of 3.803 when treated with IFN-λ2&3, and a fold-change increase of 5.027 when treated with lipoplexes (TABLE 4). This data shows that microvascular endothelial cells do not express IFN λR1 or respond to IFN-λ. However, liver epithelial cells do express IFN λR1 and show an increase in IFN λR1 gene expression in response to IFN-λ and lipoplex treatment. From this data it can be concluded that liver epithelial cells not only express IFN λR1 but are capable of responding to IFN-λ and upregulating IFN λR1 when exposed to lipoplexes.

TABLE 4
RESULTS OF qPCR OF IFNLR1 (INTERFERON
LAMBDA RECEPTOR 1) GENE EXPRESSION IN
PRIMARY HUMAN LIVER EPITHELIAL CELLS

| | | | | | 2\hat{ }-ΔΔCt |
|---|---|---|---|---|---|
| | | | | | (Relative fold- |
| | | | | | change compared |
| Treatment | ΔCt mean | ΔCt SD | ΔCt SE | ΔΔCt | to untreated) |
| IL29 | 19.476 | 0.313 | 0.128 | −1.092 | 2.132 |
| (IEN-λ1) | | | | | |
| IL28A/B | 18.641 | 0.256 | 0.105 | −1.972 | 3.803 |
| (IFN- λ2/3) | | | | | |
| Lipoplex | 18.239 | 0.355 | 0.145 | −2.330 | 5.027 |
| Untreated | 20.568 | 0.438 | 0.179 | 0 | 1.000 |

TABLE 5
RESULTS OF qPCR OF IFNLR1 GENE EXPRESSION IN PRIMARY
HUMAN LUNG MICROVASCULAR ENDOTHELIAL CELLS

| | | | | | 2\hat{ }-ΔΔCt |
|---|---|---|---|---|---|
| | | | | | (Relative fold- |
| | | | | | change compared |
| Treatment | ΔCt mean | ΔCt SD | ΔCt SE | ΔΔCt | to untreated) |
| IL29 | No Amplification | n/a | n/a | n/a | n/a |
| (IEN-λI) | Detected | | | | |
| IL28A/B | No Amplification | n/a | n/a | n/a | n/a |
| (IFN- λ2/3) | Detected | | | | |
| Untreated | No Amplification | n/a | n/a | n/a | n/a |
| | Detected | | | | |

### Example 14: Effect of IFN-λ Pre-Treatment on Doxil® Accumulation

Doxil® was added to 90% IPA/0.075 M HCl and serially diluted to obtain a standard curve of doxorubicin fluorescence. We observe a direct correlation between the amount of Doxil® in solution and doxorubicin fluorescence. The lowest level of detection from the standard curve was 2.441e-6 μg/μL of doxorubicin in 90% IPA/0.075 M HCl (FIG. 22).

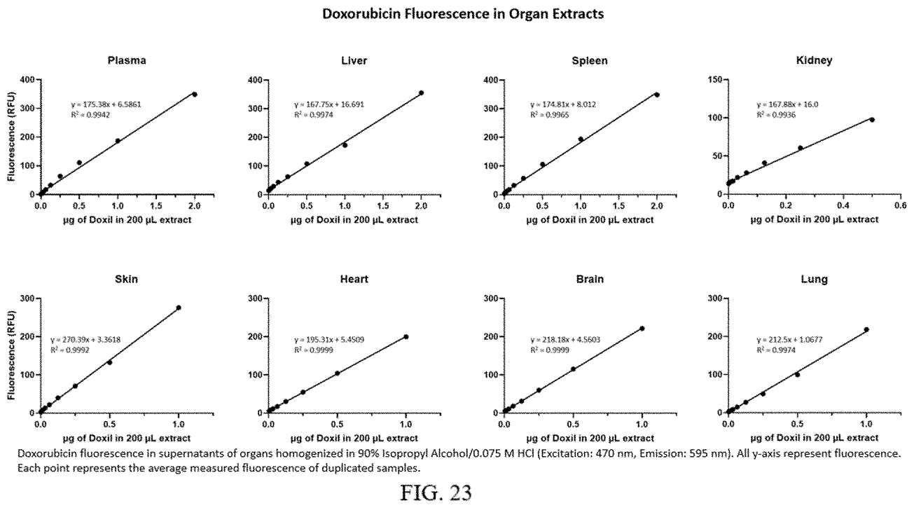

Organs and tissues from untreated mice were homogenized in 90% IPA/0.075 M HCl. Doxil® was then added to 500 μL of supernatant from centrifuged homogenates to create stock solutions of 0.01 μg/μL doxorubicin. These stock solutions were then serially diluted with the remaining homogenate supernatants to obtain standard curves of doxorubicin fluorescence in each organ homogenate. We observe a direct correlation between the amount of Doxil® in organ homogenate and doxorubicin fluorescence for each individual organ (FIG. 23).

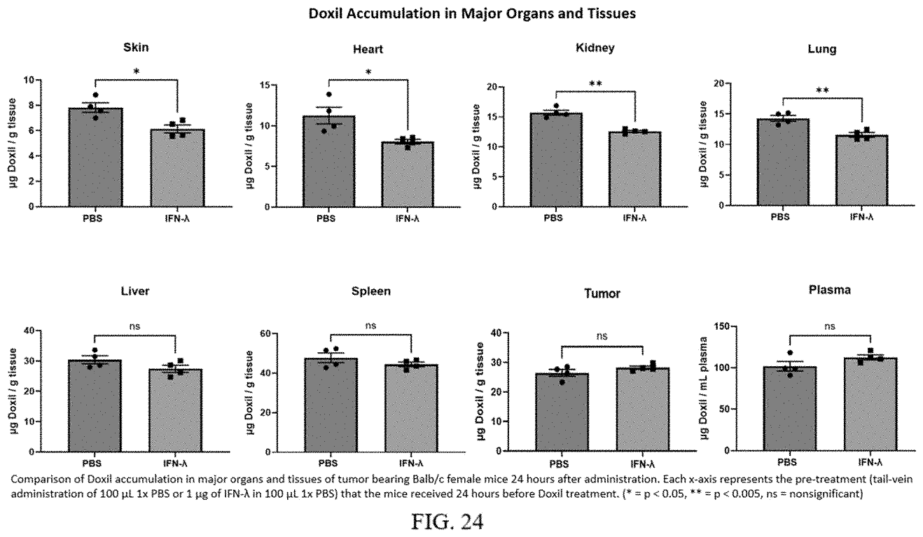

To assess the effects of IFN-λ pre-treatment on Doxil® accumulation, mice were intravenously injected with either 100 μL of 1×PBS or 1 μg of IFN-λ in 100 μL 1×PBS. Twenty-four hours after pretreatment, a 10 mg/kg dose of Doxil was administered. After an additional 24 hours following Doxil administration, tissues were harvested from experimental mice and the total Doxil accumulation in tissues was determined using the extraction method and standard curves described above. Our results demonstrate that IFN-2\ pretreatment significantly reduces Doxil accumulation in the skin, kidney, lungs, and heart when compared to mice pretreated with PBS. We also observe a reduction in Doxil accumulation for other major organs (liver, spleen). In contrast to that seen in the major organs, the tumor and plasma experienced an increase in Doxil accumulation when compared to mice pretreated with PBS, but these differences were not statistically significant (FIG. 24).

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary aspect, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular aspect disclosed as the best mode contemplated for carrying out this invention, but that the invention will include all aspects falling within the scope of the appended claims. Any combination of the above-described elements in all possible variations thereof is encompassed by the invention unless otherwise indicated herein or otherwise clearly contradicted by context.

1. A method for treating cancer in a subject, comprising predosing the subject with a composition to induce tightening of epithelial junctions in nontumor tissue, followed by administering a dose of an anti-cancer agent, wherein the predosing increases uptake or accumulation of the anti-cancer agent in tumor tissue compared to uptake or accumulation of the anti-cancer agent in nontumor tissue.

2. The method of treating cancer in a subject according to claim 1, wherein the composition to induce tightening of epithelial junctions in nontumor tissue is a composition comprising a nucleic acid and a nanoparticle mimic, an interferon λ (IFN-λ), or a combination thereof.

3. The method of treating cancer in a subject according to claim 2, wherein the nanoparticle mimic is a lipoplex composition comprising a lipid nanoparticle (NP).

4. A method for imaging a tumor in a subject, comprising predosing the subject with a composition to induce tightening of epithelial junctions in nontumor tissue, followed by administering an imaging agent, wherein the predosing increases uptake or accumulation of the imaging agent in tumor tissue compared to uptake or accumulation of the imaging agent in nontumor tissue.

5. The method for imaging a tumor in a subject according to claim 4, wherein the composition to induce tightening of epithelial junctions in nontumor tissue is a composition comprising a nucleic acid and a nanoparticle mimic, an interferon λ (IFN-λ), or a combination thereof.

6. The method for imaging a tumor according to claim 5, wherein the nanoparticle mimic is a lipoplex composition comprising a lipid NP.

7. The method of claim 3, wherein the lipid NP is formulated with asymmetric phospholipid: structural lipid: symmetric phospholipid, wherein
the asymmetric phospholipid is a sphingophospholipid or a sphingosine;
the symmetric phospholipid is one or more of DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG; and
the structural lipid is a sterol.

8. The method of claim 6, wherein the lipoplex is further formulated with PEG-ceramide.

9. The method of claim 6, wherein the NP is formulated with sphingosine, cholesterol, and DAPC, and wherein the sphingosine:cholesterol:DAPC is at a mole ratio of 3:2:5.

10. The method of claim 2, wherein the nucleic acid is one or more of a DNA, RNA, TNA, GNA, PNA, or LNA, and wherein the nucleic acid is triple-stranded, double-stranded, or single stranded.

11. The method of claim 2, wherein the composition to induce tightening of epithelial junctions in nontumor tissue is administered parenterally.

12. The method of claim 2, wherein the anti-cancer agent is a particulate agent with a hydrodynamic radius of about 2-1000 nm, a chemotherapeutic nanoparticle, contains nucleic acids, or lacks nucleic acids.

13. The method of claim 2, wherein the tumor is a solid tumor.

14. The method of claim 2, wherein the predosing is repeated prior to each dose of the anti-cancer agent, and is repeated prior to every two doses, three doses, four doses, or five doses of the anti-cancer agent.

15. The method of claim 2, wherein the predosing is repeated twice, three times, four times, or five times prior to receiving a dose of the anti-cancer agent.

16. The method of claim 2, wherein the predosing is repeated at about 10 minutes to about 24 hours.

17. The method of claim 2, wherein the predosing is administered from 5-120 minutes prior to administration of the anti-cancer agent, or the predosing is administered 3 hrs to 28 days prior to administration of the anti-cancer agent.

18. A method for treating cancer according to claim 1, wherein said predosing reduces in a subject uptake of an intravenous administration of a chemotherapeutic agent in nontumor tissues for a duration of from 3 hours to about 28 days after predosing.

19. A method for selectively reducing uptake or accumulation of an agent in nontumor tissue compared to tumor tissue of a subject comprising administering to said subject an effective amount of a composition to induce tightening of epithelial junctions in nontumor tissue, wherein the composition to induce tightening of endothelial and/or epithelial junctions in nontumor tissue is a composition comprising interferon 1 (IFN-λ), is a composition comprising a nucleic acid and a nanoparticle mimic, an interferon λ (IFN-λ) or a combination thereof.

20. The method for selectively reducing uptake or accumulation in a nontumor tissue of a subject according to claim 19, wherein the nanoparticle mimic is a lipoplex composition comprising a lipid nanoparticle (NP).
